(19) United States
(12) Patent Application Publication
(10) Pub. No.: US 2007/0219159 A1
(43) Pub. Date: Sep. 20, 2007

(54) CYCLODEXTRIN COMPOSITIONS AND METHODS OF TREATING VIRAL INFECTIONS

(75) Inventors: Kendall B. Wallace, Duluth, MN (US); Muhammad A. Khan, Duluth, MN (US); Robert M. Carlson, Duluth, MN (US); Stephen Rice, Minneapolis, MN (US); Mervin Kent Froberg, Danbury, WI (US)

Correspondence Address: ALSTON & BIRD LLP, BANK OF AMERICA PLAZA, 101 SOUTH TRYON STREET, SUITE 4000, CHARLOTTE, NC, 28280-4000, US

(73) Assignee: Regents of the University of Minnesota

(21) Appl. No.: 11/566,635

(22) Filed: Dec. 4, 2006

Related U.S. Application Data
(63) Continuation of application No. 10/394,449, filed on Mar. 21, 2003.
(60) Provisional application No. 60/366,429, filed on Mar. 21, 2002.
(60) Provisional application No. 60/456,112, filed on Mar. 19, 2003.

(51) Int. Cl. A61K 31/724

(52) U.S. Cl. 514/58; 536/103

(57) ABSTRACT

The present invention provides methods and therapeutic compositions for treating viral infections.

### CLAIM OF PRIORITY

This application claims priority under 35 U.S.C. §119(e) to U.S. Provisional Application Ser. No. 60/366429, filed Mar. 21, 2002, and to U.S. Provisional application Ser. No. ______ (Attorney Docket No. 600.594PRV; “In Vitro Activity of Beta-Cyclodextrin Against HSV-1 and HSV-2”), filed Mar. 19, 2003, which are incorporated herein by reference.

### BACKGROUND OF THE INVENTION

Some 56 million Americans have a sexually transmitted disease (STD) other than acquired immunodeficiency syndrome (AIDS). Many more people acquire STDs each year. The causative bacterial, viral, or parasitic agents for STDs are spread primarily by sexual contact. In addition, viral agents, in particular, are transmitted by others means, such as parenterally, e.g., by use of contaminated needles and syringes. STDs caused by infectious viral agents include, but are not limited to, genital herpes, which is caused by herpes simplex viruses (HSVs); AIDS, caused by human immunodeficiency virus (HIV); genital warts, caused by human papillomaviruses (HPVs); spastic paralysis and adult T cell leukemia, caused by human T-cell leukemia or lymphotropic virus type 1 (HTLV-1); and viral hepatitis, caused by hepatitis viruses, mainly hepatitis B virus (HBV) and hepatitis C virus (HCV).

It is estimated that over 4 billion U.S. dollars per year are spent worldwide on the various pharmaceuticals prescribed to treat viral STDs. For example, famciclovir (FAMVIR, Novartis), acyclovir (ZORIVAX; GlaxoSmithKline), penciclovir (Denavir), valacyclovir (VALTREX, GlaxoWellcome, Inc) and foscarnet (FOSCAVIR, AstraZeneca), are used to treat HSV-related diseases alone. These agents have been shown to speed the healing and the resolution of symptoms in both primary and recurrent episodes of genital herpes. However, the clinical use of acyclovir (ACV), the current “gold standard” of anti-herpes medications, is limited. Moreover, many side effects are associated with these anti-viral agents. Common side effects associated with the above-mentioned medications include nausea, diarrhea, and headache. In particular, foscarnet, when administered intravenously, can have several toxic effects, such as reversible impairment of kidney function or induction of seizures. Moreover, these drugs do not cure the herpes infection, but rather suppress the symptoms of the disease by inhibiting active replication of the virus.

The herpes market and the STD market in general has significant unmet medical needs including improving disease prevention, e.g., by reducing or eliminating the incidence of viral infection, e.g., by decreasing viral transmission, as well as enhancing patient compliance through improved medicine regimens. Therefore, there is a need for additional and effective preventive and therapeutic modalities against viral diseases, e.g., sexually transmitted viral diseases.

### SUMMARY OF THE INVENTION

The invention provides a method for treating a viral infection in a mammal, such as a human, comprising administering to a mammal in need of such treatment an effective amount of a cyclodextrin (CD), such as an α-cyclodextrin (α-CD), a β-cyclodextrin (β-CD), a γ-cyclodextrin (γ-CD), a derivative thereof (e.g., methyl-β-CD (MBCD)), or a pharmaceutically acceptable salt thereof For example, the CD can be β-CD. In one embodiment of the invention, the CD derivative is MBCD.

The method of the invention can be used to treat sexually transmitted diseases (STD). For example, the method can be used to treat infections caused by a wild-type herpes virus, such as HSV-1 or HSV-2, as well as a drug resistant herpes virus, e.g., an ACYCLOVIR-resistant herpes viruses. In addition, the method can be used to treat an infection caused by Epstein-Barr Virus (EBV), human papillomavirus (HPV), hepatitis virus, e.g., hepatitis B virus (HBV) or hepatitis C virus (HCV), cytomegalovirus, molluscum contagiosum virus, or a pox virus (e.g., vaccinia). The method may also be used to treat a virus infection, wherein the virus is not HIV. In addition, treatments may be directed against primary or recurrent viral infections. In one embodiment of the invention, the viral infection is caused by a herpes virus. In another embodiment, the viral infection is caused by a pox virus.

An additional anti-viral drug, e.g., famciclovir, acyclovir (ACV), valaciclovir, foscamet, penciclovir, etc., may be administered in conjunction with the CD and may enhance the therapeutic effect of the CD. In an embodiment of the invention, the additional anti-viral drug is acyclovir. An anti-retroviral agent, such as a nucleoside analogue reverse transcriptase inhibitor, a non-nucleoside analogue reverse transcriptase inhibitor, or a protease inhibitor, may also be administered in conjunction with the CD and may enhance the therapeutic effect of the CD.

Additionally, the invention provides a pharmaceutical composition comprising a CD or a pharmaceutically acceptable salt thereof. The invention also provides a pharmaceutical composition comprising CD or a pharmaceutically acceptable salt thereof and an anti-viral drug, e.g., famciclovir, acyclovir, valaciclovir, foscarnet and penciclovir, in combination with a pharmaceutically acceptable diluent or carrier.

The pharmaceutical compositions of the invention are useful for prevention (e.g., as a vaginal microbicidal agent) or medical therapy (e.g., for use in treating sexually transmitted virus infections). The invention further provides the use of a CD for the manufacture of a medicament useful for the treatment of STDs in a mammal, such as a human.

### BRIEF DESCRIPTION OF THE FIGURES

FIG. 1 depicts the effects of BCD and ACV on the yield of cell-free (solid bars) and cell-associated virus (open bars) 24 hours post-infection. Vero cells were infected with HSV-1 strain KOS1.1 at a MOI of 50, and subjected to pre (2.5 hours) and during (1.5 hours) and post (24 hours) infection treatment with BCD (7.2 mg/ml), ACV (400 μg/ml) or both. Virus titers were established for both the used media and infected cell preparation and are presented on an exponential scale as plaque forming units (PFU) per ml. For cell-free virus, titers are presented per ml of used medium, whereas for cell-associated virus, titers are calculated per ml of the virus lysate (final volume is 0.7 ml). Values are averages of two independent experiments, and error bars represent SD.

FIG. 2 depicts the effect of β-CD on ACV-resistant viruses. Monolayers of Vero cells were infected separately with HSV KOS1.1 (ACV-sensitive, wild type HSV-1) and dlsptk (an ACV-resistant, tk deletion mutant of HSV-1; Coen et al, 1989) at a MOI of 10 for one hour. Then, cells were treated with plain media (none), 20 μM ACV, or 6.4 mg/ml of β-CD for 24 hours. After treatment, equal volumes of sterile milk were added and infected cell cultures frozen. Virus lysates were prepared by three cycles of quick-freeze thawing and titration performed on Vero cells. Virus titers are expressed as PFU and each reading is in duplicate (mean +SD).

FIG. 3 depicts the effects of ACV alone (400 μg/ml) and β-CD alone (8 mg/ml) on the viability of Vero cells as monitored by the LDH release assay after 24 hours of treatments (A); and after 48 hours of treatments (B). All data represents means+SD of two separate experiments.

FIG. 4 depicts scattergrams produced by Live/Dead® Viability Assay.

FIG. 5 is a cell killing graph for Vero cells exposed to a methyl β-CD (MBCD) for 48 hours.

### DETAILED DESCRIPTION OF THE INVENTION

A number of cyclodextrins, including β-CD, are used as drug carrier molecules to modify the solubility and bioavailability and/or to reduce the associated toxicity of a number of pharmacologically important compounds. Some data indicate that β-CD has activity against HIV-1 (U.S. Patent Application Publication Nos. US 2002/0128227, US 2002/0132791; Khanna et al., 2002; and Liao et al., 2001). However, until now it was not known that β-CD had anti-viral activity against other types of viruses.

The anti-viral effect that β-CD has against herpes, vaccinia, Epstein-Barr virus and hepatitis C virus is disclosed herein.

Studies herein indicate that at the effective concentrations (5 to 10 mg/ml), the anti-HSV activity of β-CD is comparable to that of acyclovir (100 to 400 μg/ml) when tested against HSV-1 at the high MOI of 10.

There is evidence that β-CD in combination with acyclovir exhibits additive or even synergistic effects against HSV. Treatment of HSV-1 infected Vero cells with what are otherwise minimally and partially effective concentrations of either acyclovir or β-CD alone, produces a remarkable anti-viral effect when the two agents are administrated in combination at such concentrations. These results indicate a clinical benefit of developing dual regimens for treating viral infections associated with STDs.

Thus, pharmaceutical compositions, e.g., topical and other formulations, containing β-CD alone or in combination with other anti-herpes compounds like acyclovir, may prove to be highly effective formulations to prevent and treat STDs caused by herpes viruses, in particular, HSV-1 and HSV-2. Similar combinations of β-CD with anti-viral compounds known to inhibit other viruses can also be used, as well as with anti-retroviral agents, e.g., for HIV treatment (Miller et al. 1992). The additive or even synergistic action between β-CD and anti-viral agents and/or anti-retroviral agents may have effective anti-viral activity at non-toxic doses of the individual agent(s). Accordingly, the combination of β-CD and an anti-viral agent can be used to treat acyclovir-resistant and/or other drug resistant herpes virus infections.

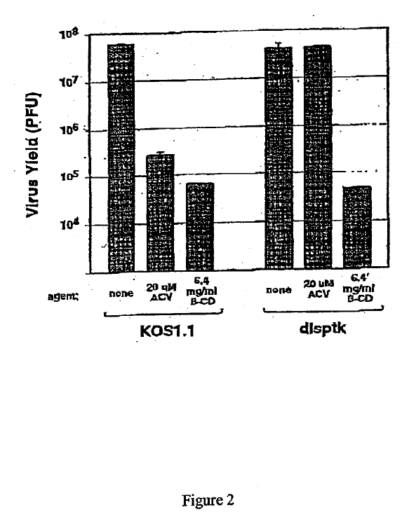

In addition, use of these combinations may be useful to treat or prevent the emergence of otherwise drug-resistant mutant viruses. For example, an acyclovir-resistant HSV has arisen through use of acyclovir and/or related drugs in the clinical treatment of herpes virus infections. Data presented herein indicates that β-CD is effective against an acyclovir-resistant HSV-1 (TK deletion mutant). Post-infection treatment with β-CD lowers the virus yield from Vero cells infected with either wild-type HSV-1 strain (KOS1.1) or a TK deletion mutant (dlsptk) of HSV-1 (FIG. 2) to similar levels. Whereas acyclovir and β-CD reduce virus yields from cells infected with wild-type HSV-1 to similar levels when applied separately, acyclovir treatment alone does not lower the virus yield from cells infected with dlsptk.

Studies on the mechanism of action of β-CD indicate that it acts at an early stage of the HSV infection cycle. While β-CD may not inhibit viral entry, initial studies indicate that it affects the expression of immediate early (IE) genes. In contrast, acyclovir is initially phosphorylated by viral (HSV) thymidine kinase, and later by additional cellular kinases to form acyclovir triphosphate. This activated drug interferes with HSV DNA polymerase and viral DNA replication in infected cells, which occurs after expression of IE genes, i.e., later in the infection cycle.

In addition to having anti-viral activity against herpes virus, the inventors have discovered that CD and derivatives thereof are effective against vaccinia virus, EBV and HCV.

I. Definitions

Unless defined otherwise, all technical and scientific terms used herein have the same meaning as commonly understood by one of ordinary skill in the art to which this invention belongs. For purposes of the present invention, the following terms are defined below.

“Cyelodextrin” refers to a cyclic oligosaccharide consisting of at least five saccharide units (e.g., glucopyranose units). For example, the term “cyclodextrin” includes a cyclic molecule containing six or more α-D-glucopyranose units linked at the 1,4 positions by α linkages, as in amylose, as well as a cyclic molecule containing seven α-D-glucopyranose units, as in cycloheptaamylose. The term “cyclodextrin” includes any of the known cyclodextrins, such as unsubstituted cyclodextrins containing from six to twelve glucose units. Thus, the term “cyclodextrin” includes at least beta-cyclodextrin (β-CD or BCD), which is commercially available (e.g., product no. C-4805 from Sigma-Aldrich Corp., St. Louis, Mo., USA, cell culture grade β-CD (Schardinger β-Dextrin; Cycloheptaamylose)), as well as alpha-cyclodextrin (α-CD or ACD) and gamma-cyclodextrin (γ-CD or GCD) and/or their derivatives and/or mixtures thereof The α-cyclodextrin consists of six glucose units, the β-cyclodextrin consists of seven glucose units, and the γ-cyclodextrin consists of eight glucose units arranged in donut-shaped rings. The term “derivative” of cyclodextrin is meant to include a cyclodextrin molecule wherein some of the OH groups are converted to OR groups. For example, cyclodextrin derivatives include those substituted with lower alkyl groups such as methylated cyclodextrins and ethylated cyclodextrins, wherein R is a methyl or an ethyl group. Lower alkyls contain from 1 to 6 carbon atoms and may be straight chain or branched. In addition, cyclodextrin derivatives include those with hydroxyalkyl substituted groups, such as hydroxypropyl cyclodextrins and/or hydroxyethyl cyclodextrins, wherein R is a —CH₂ —CH(OH)₂—CH₃ or a —CH₂CH₂—OH group. Substitution may occur at some or all of the hydroxyl groups. By way of example, a derivative of β-cyclodextrin is methyl-β-cyclodextrin (MBCD). The term “methyl-β-cyclodextrin” refers to a β-cyclodextrin having hydroxyl sites substituted by methoxy groups to varying degrees. For example, MBCD can be totally saturated, i.e., 80-100% substituted. Alternatively, the mean degree of substitution can be about 1.5-2.1 methyl units/glucose, i.e., approximately 25-33% substituted. Methyl-β-cyclodextrin useful in the invention is commercially available (e.g., product no. C-4555, Sigma).

“Derivatives” of cyclodextrin also include cyclodextrin derivatives such as hydroxypropyl and sulfobutyl ether cyclodextrins and others. Such derivatives are described for example, in U.S. Pat. Nos. 4,727,064 and 5,376,645. Hydroxypropylated β-cyclodextrins (HPBCD) are commercially available (e.g., 2-hydropropyl-β-cyclodextrin, product no. C-0926, Sigma); as are Hydroxypropylated α-cyclodextrins (HPACD) (e.g., CAVASOL® W6 HP, Wacker Biochem Corp. USA, Eddyville, Iowa 52553) and hydroxypropylated γ-cyclodextrins (HPGCD) (e.g., CAVASOL® W8 HP, Wacker Biochem Corp.). Sulfobutyl-ether-β-cyclodextrin are also commercially available. Additional cyclodextrin derivatives are disclosed, for example, in U.S. Pat. No. 6,001,343.

“Treating” as used herein refers to ameliorating at least one symptom of, curing and/or preventing the development of a given disease or condition. By “preventing” is meant attenuating or reducing the ability of a virus to cause infection or disease, e.g., by affecting a post-entry viral event. For example, “preventing” can refer to attenuating the primary infection or transmission of the virus.

A “thymidine kinase-deficient” virus is a virus comprising a disrupted nucleic acid sequence encoding thymidine kinase (TK), such that the thymidine kinase activity of the virus is reduced or eliminated as compared to a corresponding wild-type or non-TK-deficient virus.

A composition is said to be “pharmacologically acceptable” if its administration can be tolerated by a recipient patient. Such an agent is said to be administered in a “therapeutically effective amount” if the amount administered is physiologically significant. A composition of the present invention is physiologically significant if its presence results in a detectable change in the physiology of a recipient patient, e.g., ameliorates at least one symptom associated with a viral infection, prevents or reduces the rate transmission of at least one viral agent.

II. Exemplary Viruses Amenable to Treatment by the Methods of the Invention

The following is a non-inclusive list of exemplary viruses amenable to the methods of the invention. Other viruses, including other sexually-transmitted viruses, may also be treated by the methods disclosed herein.

A. Herpes Virus

Over 40 million Americans suffer from cold sores (a common form of oral herpes) which are caused by HSV-1. HSV-1 can be transmitted through oral secretions, e.g., during kissing or by using contaminated food preparations and utensils. HSV-1 is also responsible for causing 5% to 10% of genital herpes. Initial oral herpes infections with HSV-1 usually occur in childhood, and thus are not classified as a STD.

HSV-2 causes the majority of genital herpes, one of the fastest growing STDs in the world. Roughly 86 million people worldwide are infected with HSV-2, of which 22 million display symptoms of painful genital blisters and sores with typically 5 to 8 outbreaks annually. Only 2.6% of those afflicted with genital herpes have symptomatic infection. HSV-2 can be transmitted through direct personal contact and/or through oral or genital secretions, regardless of the presence of the symptoms.

Primary herpes virus infection occurs through a break in the mucous membranes of the mouth or throat, via the eye or genitals, or directly via minor abrasions in the skin. Because of the global distribution of HSV-1, most individuals are infected by 1-2 years of age. Initial infection is usually asymptomatic, although there may be minor local vesicular lesions. Local multiplication ensues, followed by viremia and systemic infection. A life-long latent infection with periodic reactivation follows.

During an initial (primary) infection, the herpes virus enters peripheral sensory nerves and migrates along axons to sensory nerve ganglia in the central nervous system (CNS), escaping an immune response. During latent infection of nerve cells, viral DNA is maintained as an episome (i.e., it is not integrated). There is, however, limited expression of specific virus genes required for the maintenance of latency.

Outbreaks are triggered by various disturbances, such as physical trauma, e.g., injury, ultraviolet light, hormones, stress, surgical trauma, or psychological trauma, e.g., emotional stress, which affect the immune system or hormonal balance.

Reactivation of latent virus leads to recurrent episodes of the disease. During recurrent infections, virus is reactivated and travels down sensory nerve ganglia to the surface of the body, re-infecting the skin and replicating, which causes tissue damage. Although painful, most recurrent infections resolve spontaneously, usually to reoccur later. More serious conditions include herpetic keratitis (ulceration of cornea due to repeated infections that can lead to blindness) and encephalitis, which is very rare and often fatal.

Genital herpes is usually transmitted sexually and hence its incidence can be reduced or eliminated by use of appropriate vaginal anti-viral agents, such as a cyclodextrin, e.g., β-CD.

Epstein-Barr virus, frequently referred to as EBV, is another member of the herpesvirus family and one of the most common human viruses. The virus occurs worldwide, and most people become infected with EBV sometime during their lives. When infection with EBV occurs during adolescence or young adulthood, it causes infectious mononucleosis 35% to 50% of the time. Symptoms of infectious mononucleosis are fever, sore throat, and swollen lymph glands. Sometimes, a swollen spleen or liver involvement may develop. Heart problems or involvement of the central nervous system can occur. EBV also establishes a lifelong dormant infection in some cells of the body's immune system. A late event in a very few carriers of this virus is the emergence of Burkitt's lymphoma and nasopharyngeal carcinoma. EBV appears to play an important role in these malignancies, but is probably not the sole cause of disease.

Currently, there is no specific treatment options available for infectious mononucleosis, other than treating the symptoms.

B. Human Papillomaviruses

In recent years, HPVs have been shown to be a group of the most common sexually transmitted viruses in the U.S. (Jay and Moscicki, 2000; Kaiser Family Foundation, 2000). Up to 20 million Americans are currently infected with sexually transmitted HPVs, which are double stranded DNA viruses that cause genital warts (condylomata acuminata) (see, in general, Howley and Lowy, 2001, and Lowy and Howley, 2001).

It is estimated that about 75 percent of adult population has been infected with genital HPV at some point in their lives (Cates, 1999). While there are more than 65 types of the HPV, over 90 percent cases of genital warts are due to HPV types 6 and 11 (Jay and Moscicki, 2000). However, infection with specific types of HPV (mainly types 16, 18, 31, and 45) can lead to neoplastic changes in genital epithelia resulting in cancers of lower genital tract, including commonly occurring cervical carcinomas of women. Moreover, scientists have found association between several types of HPV and development of a number of cancers, including oral cancer and cancers of the anogenital region, such as cervical, vulvar, anal, and penile cancer. Because of the contagious spread and carcinogenic potential, HPV infections require treatment.

Depending on factors such as their size and location, genital warts are treated in several ways. Undiluted trichloroacetic acid preparation (TCA) can be applied to the infected area and washed off several hours later. An alternative treatment is a 20 percent podophyllin solution, which is applied to the affected area and later washed off. Pregnant women should not use podophyllin because it is absorbed by the skin and may cause birth defects in babies. Applications of five percent 5-fluorouracil cream may also be prescribed, although, as with podophyllin, it should be avoided during pregnancy. In addition, small warts can be removed by destructive methods, e.g., cryosurgery (freezing) or electrocautery (burning). Surgery is occasionally needed to remove large warts that have not responded to other treatment. Side effects that may occur with conventional treatments include pain, burning, inflammation, skin erosion, scarring, and erythema.

A new treatment of external genital and perianal warts, Aldara (imiquimod) cream, has recently been approved by the FDA. Aldara cream is the newest in a class of drugs called immune response modifiers and represents the first new therapeutic approach to genital warts in five years.

The drug alpha interferon (αINF) is used when warts have recurred after removal by traditional means. In studies supported by NIAID and others, investigators found that interferon treatment eliminated the warts in about half the patients. For some patients, a second course of treatment may be necessary.

Although these treatments can eliminate the warts, they do not cure the disease, i.e., warts often reappear after treatment.

C. Hepatitis Viruses

Hepatitis B is a sexually transmitted disease caused by hepatitis B virus (HBV). Chronic infections can lead to severe liver damage (cirrhosis) and liver cancer (hepatocellular carcinoma). Hepatitis C is emerging as a serious liver disease, with a significantly higher risk for IV drug abusers and sexually promiscuous individuals. This disease is caused by hepatitis C virus (HCV), which unlike HBV, establishes chronic infections regardless of the age of infected persons and hence has a much higher potential to cause cirrhosis and hepatocellular carcinoma (See, in general, Major et al., 2001).

D. Human Cytomegaloviruses and Molluscum Contagiosum Virus

In addition to herpes virus, HPVs, and hepatitis viruses, which are capable of causing diseases in healthy individuals (primary pathogens), a number of other viruses capable of sexual transmission usually cause opportunistic infections, e.g., human cytomegaloviruses (HCMV) and molluscum contagiosum virus (MCV). In general, viruses like these become clinically significant when presented with other complications, usually in immunocompromised persons, for example, patients suffering from AIDS or other forms of immunodeficiencies, or patients on therapy for different types of transplantation or cancer.

HCMV causes one of the most common and difficult opportunistic infections in immunocompromised patients. The condition can result from primary infection, recurrence by the latent virus reactivation, or re-infection with a new strain of virus in otherwise previously infected persons. In such circumstances, diagnosis is hard to establish because besides demonstrating the presence of virus (lab detection of virus), its etiology has to be established for the given condition (i.e., if CMV is causing the pathology). HCMV is frequently involved in retinitis in the AIDS patients. In addition to the horizontal route, HCMV causes the most frequent congenital infection in humans (vertical transmission), both without (asymptomatic) or with clinical symptoms (symptomatic disease) indicative of multiple organ involvement. In addition, individuals born with such infections commonly develop sensorineural deafness (CNS sequelae). HCMV is also considered as the leading cause of brain damage in children (see generally Mocarski and Courcelle, 2001 and Pass, 2001).

MCV is a poxvirus that causes dermal lesions (noninflamed skin papules) on various parts of the body, including the torso area in children and anogenital area in persons who engage in anogenital sex. A typical lesion consists of a localized mass of hypertrophied and hyperplastic epidermis extending down into the underlying dermis, but without breaking the basement membrane and projecting above the adjacent skin as a visible tumor. These lesions may last from 2 weeks to 2 years, and cropping may occur as a consequence of multiple simultaneous infections or by localized mechanical spread. MCV caused lesions may be quite persistent and disfiguring in persons suffering from AIDS. Transmission of the virus is through direct contact and through body fluids (see, in general, Esposito and Fenner, 2001).

E. Pox Virus

In addition to MCV, described herein, pox viruses amenable to the methods of the invention include vaccinia, smallpox virus (variola), cowpox, monkey pox, pseudocowpox and Orf (contagious pustular dermatitus) virus. Orf has been placed in the genus Parapoxvirus of the poxviruses. Additional human pathogens among poxviruses include yabapox virus, tanapox virus, and molluscum contagiosum virus, which is described in more detail herein.

Poxviruses are large, brick-shaped viruses about 300×200 nm. They have a double-stranded DNA genome (about 200 Kb) enclosed within a core that is flanked by two lateral bodies. The surface of the virus particle is covered with filamentous protein components. The entire particle is enclosed in an envelope derived from the host cell membranes.

Laboratory diagnosis of pox viruses may be undertaken by electron microscopy of negatively stained vesicle fluid or lesion material. Some pox viruses can be cultured on the chorio-allantoic membrane of chick embryos, where they form pocks, and some can be isolated by cell-culture.

Vaccinia, which has been used for immunization against smallpox, is a genetically distinct type of pox virus which grows readily in a variety of hosts. In humans it causes a localized pustule with scar formation. In immuno-compromised persons or eczematous persons it sometimes caused a severe generalized vaccinia infection.

III. Anti-Vial Agents of the Invention

In addition to cyclodextrins, agents useful in the practice of the invention include any agent known to the art that is useful for treating a viral infection.

For example, acyclovir (ACV) is a nucleoside analogue used clinically for early infections of a number of herpes viruses including HSV-1, HSV-2, and varicella-zoster virus (VZV). It has a shorter half-life in cells and requires a longer course of treatment than famciclovir, discussed below. Acyclovir exerts its potent anti-herpes effect through interfering with viral DNA polymerase and viral DNA replication (chain termination). While acyclovir is well tolerated, its clinical use is limited. Topical acyclovir must be applied more than five times per day to be effective. Intravenous acyclovir is sometimes needed for severe herpes infections, which often involve the brain, eyes, and lungs. Such complications typically develop in immunocompromised individuals as a consequence of the unchecked virus replication and invasion of the respective tissues/organs.

Another anti-viral agent is foscarnet, used for acyclovir-resistant HSV in immunocompromised patients. It inhibits replication of all known herpes viruses. However, many side effects are associated with the use of foscamet. For example, when administered intravenously, the drug can exhibit several toxic effects such as reversible impairment of kidney function or induction of seizures. Given these serious side effects, use of foscamet is reserved for treating severe and/or drug-resistant herpes infections.

Famciclovir (double-valine ester of penciclovir) is an anti-viral agent that undergoes rapid biotransformation in the body to release valine esters and produce high concentrations of penciclovir in the plasma.

Compared to acyclovir, penciclovir has an additional CH₂OH group. Like acyclovir, it is acquired and initially phosphorylated by viral thymidine kinase (tk) in the HSV-infected cells. However, the final active inhibitor, penciclovir triphosphate has a longer intracellular half life (12 hours) than acyclovir triphosphate (ACVTP; half life of 2.5 hours).

TK mutants of HSV exhibiting acyclovir resistance are also resistant to penciclovir and famciclovir because of the requirement of the viral enzyme for the phosphorylation of penciclovir.

For a review of these and other anti-viral agents in clinical use, see Crumpacker, 2001.

IV. Dosages, Formulations and Routes of Administration of the Anti-Viral Agents of the Invention

The pharmaceutical compositions of the invention can be administered to a mammalian host, such as a human patient in a variety of forms adapted to the chosen route of administration, i.e., orally or parenterally, by intravenous, intramuscular, topical or subcutaneous routes.

Thus, the present compositions may be systemically administered, e.g., orally, in combination with a pharmaceutically acceptable vehicle such as an inert diluent or an assimilable edible carrier. They may be enclosed in hard or soft shell gelatin capsules, may be compressed into tablets, or may be incorporated directly with the food of the patient's diet. For oral therapeutic administration, the active compound may be combined with one or more excipients and used in the form of ingestible tablets, buccal tablets, troches, capsules, elixirs, suspensions, syrups, wafers, and the like. Such compositions and preparations should contain at least 0.1% of active compound. The percentage of the compositions and preparations may, of course, be varied and may conveniently be between about two to about 60% of the weight of a given unit dosage form. The amount of active compound in such therapeutically useful compositions is such that an effective dosage level will be obtained.

The tablets, troches, pills, capsules, and the like may also contain the following: binders such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, fructose, lactose or aspartame or a flavoring agent such as peppermint, oil of wintergreen, or cherry flavoring may be added. When the unit dosage form is a capsule, it may contain, in addition to materials of the above type, a liquid carrier, such as a vegetable oil or a polyethylene glycol. Various other materials may be present as coatings or to otherwise modify the physical form of the solid unit dosage form. For instance, tablets, pills, or capsules may be coated with gelatin, wax, shellac or sugar and the like. A syrup or elixir may contain the active compound, sucrose or fructose as a sweetening agent, methyl and propylparabens as preservatives, a dye and flavoring such as cherry or orange flavor. Of course, any material used in preparing any unit dosage form should be pharmaceutically acceptable and substantially non-toxic in the amounts employed. In addition, the active compound may be incorporated into sustained-release preparations and devices.

In cases where compounds are sufficiently basic or acidic to form stable nontoxic acid or base salts, administration of the compounds as salts may be appropriate. Examples of pharmaceutically acceptable salts are organic acid addition salts formed with acids which form a physiological acceptable anion, for example, tosylate, methanesulfonate, acetate, citrate, malonate, tartarate, succinate, benzoate, ascorbate, α-ketoglutarate, and α-glyccrophosphate. Suitable inorganic salts may also be formed, including hydrochloride, hydrobromide, sulfate, nitrate, bicarbonate, and carbonate salts.

Pharmaceutically acceptable salts may be obtained using standard procedures well known in the art, for example by reacting a sufficiently basic compound such as an amine with a suitable acid affording a physiologically acceptable anion. Alkali metal (for example, sodium, potassium or lithium) or alkaline earth metal (for example calcium) salts of carboxylic acids can also be made.

The active compound may also be administered intravenously or intraperitoneally by infusion or injection. Solutions of the active compound or its salts can be prepared in water, optionally mixed with a nontoxic surfactant. Dispersions can also be prepared in glycerol, liquid polyethylene glycols, triacetin, and mixtures thereof and in oils. Under ordinary conditions of storage and use, these preparations contain a preservative to prevent the growth of microorganisms.

The pharmaceutical dosage forms suitable for injection or infusion can include sterile aqueous solutions or dispersions or sterile powders comprising the active ingredient, which are adapted for the extemporaneous preparation of sterile injectable or infusible solutions or dispersions, optionally encapsulated in liposomes. In all cases, the ultimate dosage form should be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof The proper fluidity can be maintained, for example, by the formation of liposomes, by the maintenance of the required particle size in the case of dispersions or by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars, buffers or sodium chloride. Prolonged absorption of the injectable compositions can be brought about by the use in the compositions of agents delaying absorption, for example, aluminum monostearate and gelatin.

Sterile injectable solutions are prepared by incorporating the active compound in the required amount in the appropriate solvent with various of the other ingredients enumerated above, as required, followed by filter sterilization. In the case of sterile powders for the preparation of sterile injectable solutions, the preferred methods of preparation are vacuum drying and the freeze drying techniques, which yield a powder of the active ingredient plus any additional desired ingredient present in the previously sterile-filtered solutions.

For topical administration, the present compositions may be applied in pure form, i.e., when they are liquids. However, it will generally be desirable to administer them to the skin as compositions or formulations, in combination with a dermatologically acceptable carrier, which may be a solid or a liquid. In one embodiment, a composition of the present invention is administered to vaginal skin as a cream, gel, ointment and the like, e.g., as a vaginal microbicide.

Useful solid carriers include finely divided solids such as talc, clay, microcrystalline cellulose, silica, alumina and the like. Useful liquid carriers include water, alcohols or glycols or water-alcohol/glycol blends, in which the present compositions can be dissolved or dispersed at effective levels, optionally with the aid of non-toxic surfactants. Adjuvants such as fragrances and additional antimicrobial agents can be added to optimize the properties for a given use. The resultant liquid compositions can be applied from absorbent pads, used to impregnate bandages and other dressings, or sprayed onto the affected area using pump-type or aerosol sprayers.

Thickeners such as synthetic polymers, fatty acids, fatty acid salts and esters, fatty alcohols, modified celluloses or modified mineral materials can also be employed with liquid carriers to form spreadable pastes, gels, ointments, soaps, and the like, for application directly to the skin of the user.

Examples of useful dermatological compositions which can be used to deliver the compositions of the invention to the skin are known to the art; for example, see Jacquet et al. (U.S. Pat. No. 4,608,392), Geria (U.S. Pat. No. 4,992,478), Smith et al. (U.S. Pat. No. 4,559,157) and Wortzman (U.S. Pat. No. 4,820,508).

Useful dosages of the compositions of the invention can be determined by comparing their in vitro activity, and in vivo activity in animal models. Methods for the extrapolation of effective dosages in mice, and other animals, to humans are known to the art; for example, see U.S. Pat. No. 4,938,949.

Generally, the concentration of the composition(s) of the invention in a liquid composition, such as a lotion, will be from about 0.1-25 wt-%, preferably from about 0.5-10 wt-%. The concentration in a semi-solid or solid composition such as a gel or a powder will be about 0.3-5 wt-%, preferably about 0.5-2.5 wt-%.

The amount of the composition required for use in treatment will vary not only with the particular salt selected but also with the route of administration, the nature of the condition being treated and the age and condition of the patient and will be ultimately at the discretion of the attendant physician or clinician.

In general, however, a suitable dose will be in the range of from about 0.5 to about 100 mg/kg, e.g., from about 10 to about 75 mg/kg of body weight per day, such as 3 to about 50 mg per kilogram body weight of the recipient per day, preferably in the range of 6 to 90 mg/kg/day, most preferably in the range of 15 to 60 mg/kg/day.

The composition is conveniently administered in unit dosage form; for example, containing 5 to 1000 mg, conveniently 10 to 750 mg, most conveniently, 50 to 500 mg of active ingredient per unit dosage form.

Ideally, the active ingredient should be administered to achieve peak plasma concentrations of the active compound of from about 0.5 to about 75 μM, preferably, about 1 to 50 μM, most preferably, about 2 to about 30 μM. This may be achieved, for example, by the intravenous injection of a 0.05 to 5% solution of the active ingredient, optionally in saline, or orally administered as a bolus containing about 1-100 mg of the active ingredient. Desirable blood levels may be maintained by continuous infusion to provide about 0.01-5.0 mg/kg/hr or by intermittent infusions containing about 0.4-15 mg/kg of the active ingredient(s).

The desired dose may conveniently be presented in a single dose or as divided doses administered at appropriate intervals, for example, as two, three, four or more sub-doses per day. The sub-dose itself may be further divided, e.g., into a number of discrete loosely spaced administrations; such as multiple inhalations from an insufflator or by application of a plurality of drops into the eye.

The compositions of the invention can also be administered in combination with other therapeutic agents that are effective to treat viral infections.

The invention will now be illustrated by the following non-limiting Examples.

### EXAMPLE 1

The activity of aqueous formulations of β-CD against strains of HSV-1 and HSV-2 was examined in an in vitro model using the Vero cell line. These studies indicated that β-CD is an effective anti-viral agent.

Materials and Methods

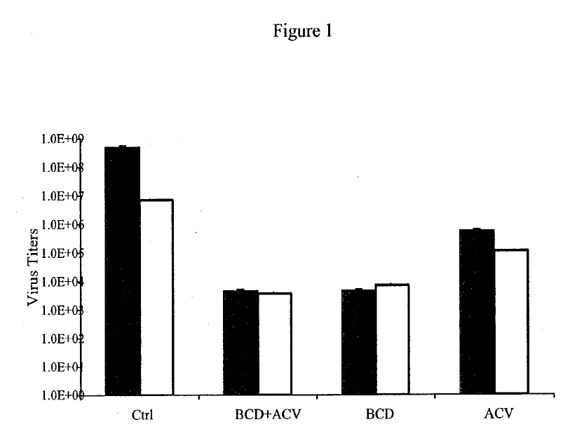

Antiviral assays. The effect of β-CD, ACV, and ACV+β-CD on the replication of HSV-1 strain KOS1.1 (FIG. 1) was studied by the following assays.

Cultures of Vero cells (from ATCC) were started in six-well plates by seeding 4-6×10⁵ cells in MEM supplemented with 5% FBS medium per well and incubating in a 5% CO₂, humidified incubator at 37° C. Monolayers of 90% or more conflucnce that formed after 16-24 hours of incubation were used in the assays. Where indicated, pre-infection treatments were initiated by aspirating used medium and adding 2 ml of medium containing the respective formulations (stock solutions of compounds added to 5× MEM to yield 1× MEM-5% FBS medium containing indicated concentration of the treatment substance). Both negative (no virus) and positive virus controls (virus infection without treatment) received growth medium only (5× diluted to 1× MEM-5% FBS). Plates were returned to the incubator and pre-treatments lasted from 3 to 5 hours. Media were then aspirated and cells were washed once with fresh 1× MEM-5% FBS before infecting with the virus.

To infect cells, frozen vials of virus (HSV-1 or HSV-2) were quickly thawed in a 37° C. water bath, and the indicated virus dilutions prepared in 1× MEM-5% FBS at room temperature. Used medium was aspirated, cells were inoculated with 1 ml of virus dilutions, and the plates returned to the incubator for 1.5-2 hours. Unless otherwise mentioned, no test formulations were added and hence no treatments done during the infection. Negative control wells received equal volumes of fresh 1× MEM-5% FBS. Virus dilutions were aspirated and post-infection treatments started by adding 2 ml of medium containing the respective formulation, while controls received growth medium only. These treatments lasted from one to several days and the wells received fresh media every 48 hours.

To harvest used media for titration of cell-free virus produced in different treatments, 350 μl aliquots were withdrawn at a given time post infection (PI), placed on ice, quickly frozen by placing tubes in liquid nitrogen for 2-3 minutes and stored at −80° C. To make cell-associated virus preparations, cells were washed with Dulbecco's PBS, treated with trypsin, centrifuged, and resuspended in fresh 1× MEM-5% FBS. A second cycle of centrifugation and aspiration was performed to ensure washing, after which cells were finally resuspended in 700 μl of fresh 1× MEM-5% FBS. To release virus, infected cells were subject to three quick freeze-thaw cycles using liquid nitrogen and a water bath at 37° C. Virus lysates were cleared by centrifugation, and the supernatants containing virus were collected, frozen quickly and stored at −80° C. for titration at a later date.

Virus titration. Both cell-free and cell-associated virus preparations of HSV-1 strain KOS1.1 obtained from the antiviral assays (see above) were quantitated by plaque formation on Vero cells utilizing a modified agarose overlay method (Federoff 1998). Vero cells were grown to confluence in six-well plates. Frozen samples from antiviral assays were thawed, diluted, and 1 ml of different dilutions applied to wells after aspirating medium. Plates were incubated at 37° C. for 1.5-2 hours. Virus dilutions were aspirated, 0.15 ml of fresh medium added quickly to each well, and 2 ml of a 1:1 dilution of preheated 2% low melting temperature agarose in water (SeaPlaque® agarose, BMA, Rockland, Me.) and 2× MEM-FBS held at 42° C. added per well. After gel formation, plates were incubated for 48 hours and 0.5 ml of a 1:100 dilution of 0.33% Neutral Red Solution (Sigma) in 1×-MEM 5% FBS was added to each well. Plates were then wrapped in aluminum foil and incubated further for 1 to 3 days. By that point the plaques had attained good size and could be seen by the unaided eye as clear hollows over the reddish background. Counting was performed on wells containing 10 to 100 plaques.

Effect of β-CD on ACV-resistant viruses. Monolayers of Vero cells were infected separately with HSV KOS1.1 (ACV-sensitive, wild type HSV-1) and dlsptk (an ACV-resistant, tk deletion mutant of HSV-1) at MOI of 10 for 1 hour. Then, cells were treated with plain media, treatment media containing 20 μM ACV, or 6.4 mg/ml β-CD for 24 hours. After treatment, equal volumes of sterile milk were added and infected cell cultures frozen. Virus lysates were prepared and titered on Vero cells as described above.

β-CD and ACV combined formulation has a synergistic anti-viral effect. To test the antiviral effects of β-CD in combination with acyclovir, two 6-well plates containing monolayers of Vero cells were treated with different formulations (pre-infection treatment for about 2.5 hours). Cells were then infected with HSV-1-KOS1.1 at a MOI of 2 for more than 90 minutes, subjected to the following post-infection treatments. In six-well plate ‘A’, two wells (numbers 1 and 4) of infected cells were treated with 4.5 mg/ml of β-CD; two additional wells (numbers 2 and 5) of infected cells were treated with 300 μg/ml of acyclovir; and two additional wells (numbers 3 and 6) of infected cells were treated with acyclovir plus β-CD at the final concentrations of 300 μg/ml and 4.5 mg/ml, respectively. In six-well plate ‘B’, two wells (numbers 1 and 4) of infected cells were treated with 4.95 mg/ml of β-CD; two additional wells (numbers 2 and 5) of infected cells were treated with 300 μg/ml of acyclovir; and two additional wells (numbers 3 and 6) of infected cells were treated with acyclovir plus β-CD at the final concentrations of 250 μg/ml and 4.95 mg/ml, respectively.

Results

FIG. 1 illustrates the potent anti-herpes activity of β-CD in comparison with acyclovir. The data represent the titer (count) of cell-free and cell-associated virus present in Vero cells 24 hours after being infected with a high MOI of the herpes virus. Both cell-free and cell-associated viruses are reduced to a greater extent by β-CD than by a fairly high concentration of acyclovir. At the concentrations of β-CD used in this experiment, combining acyclovir with β-CD adds no apparent advantage to the effect of β-CD alone.

FIG. 2 demonstrates that β-CD is effective against acyclovir -resistant viruses. While acyclovir was able to lower the yield of acyclovir-sensitive HSV KOS1.1, but not of the acyclovir -resistant mutant, β-CD lowered the yield of both HSV KOS1.1 and acyclovir -resistant dlsptk virus (tk deletion mutant of HSV-1 (Coen et al., 1989)). The observation that β-CD is as effective against the acyclovir-resistant virus as it is against the wild type HSV KOS1.1 is significant. Thus, the activity and potency of β-CD is not altered by the acyclovir resistance, suggesting a different mode of action.

Following the titration experiments using acyclovir or β-CD alone to protect Vero cells against virus infection, concentrations of the two compounds were chosen that exhibited borderline protection against MOI of 2 with this virus. By using two such sub-optimal concentrations of β-CD (final concentrations of 4.5 and 4.95 mg/ml) that conferred marginal antiviral protection upon Vero cells, it was found that in combination with 250 to 300 μg/ml of acyclovir, β-CD enhanced the degree of protection compared to acyclovir alone (at both concentrations, but mainly 4.95 mg/ml of β-CD). These results confirm a clinical benefit of using dual drug regimens for treating HSV infections.

### EXAMPLE 2

Effect of β-CD on Cell Viability

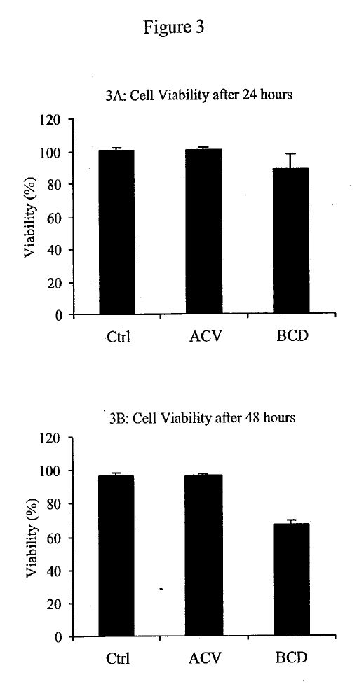

The effects of different formulations of β-CD on cell viability were studied using Vero cell line and are presented in FIG. 3.

Materials and Methods

Cell viability assay. Cell viability was determined fluorometrically by estimating the release of LDH into the media (Moran and Schnellman, 1996). LDH activity was based on the reduction of pyruvate to lactate. The concomitant oxidation of NADH results in a decrease in the fluorescence emission at 450 nm with excitation wavelength 355 nm, and the rate of disappearance of NADH is indicative of LDH activity.

To study the effects of formulations on viability of preformed monolayers, 3 to 4×10⁶ Vero cells were added to 1× MEM-5% FBS in T₇₅ flasks. After 24 hours of incubation, monolayers formed exhibiting about 85-95% confluence. Used media were replaced with 15 ml of treatment media, containing 5× MEM-FBS diluted to 1× with water (control), acyclovir stock (400 μg/ml), or β-CD stock (8 mg/ml in water). After 24 or 48 hours, media were collected and assayed for LDH. Cells were then treated with trypsin, resuspended in fresh 1× MEM-5% FBS, kept at 37° C., 5% CO₂ atmosphere, and quickly assayed for LDH. Fresh 1× MEM-5% FBS was used as blank for all assays.

Reaction solution was made fresh for each experiment by mixing 0.4 ml of 16.2 mM pyruvate with 10 ml of 0.2 mM NADH in phosphate buffer (pH 7.5). 200 μl/well of this solution was added to the required number of wells in a 96-well plate. 5 μl of either used medium or supernatant from cell suspensions was added and fluorometric studies performed. In addition to measuring LDH present in the used media or in the cell suspensions, total LDH present in cells and medium was also assayed after lysing cells with 350 μM digitonin. The amount of LDH calculated for either condition was then compared with and expressed as a percentage of the total LDH present.

Results

FIG. 3 illustrates the effect of β-CD on cell viability at both 24 and 48 hours of Vero cells in culture. At this concentration of β-CD, there is some (30-40%) decrease in cell viability at 48 hours. Acyclovir caused no drug-related cell killing in these cultures. This cytotoxicity has been reported previously for β-CD when administered by injection and limits the utility of this drug-delivery vehicle for systemic applications. For this reason, application of β-CD as an antiviral agent may be most successful for topical applications.

### EXAMPLE 3

Mechanism of Action of β-CD

Following the establishment of the anti-HSV activity of β-CD, research efforts were directed towards exploring the mechanism of action of this activity. Using a HSV-1 based vector d27-lacZ1 (Rice and Knipe, 1990), which expresses the reporter gene β-galactosidase early in the infection cycle, studies were initiated to decipher the molecular mechanism(s) involved in exerting the antiviral activity of β-CD against HSV.

Construction of the replication-defective d27-lacZ1 virus has been described (Rice and Knipe, 1990). Briefly, molecular manipulations introduced an intact lacZ gene in frame with the coding sequence of a partially deleted ICP27 gene (an IE gene), and homologous recombination substituted the corresponding region of the wild type HSV-1 genome with this modified gene region. d27-lacZ1 virus expresses an ICP27-β-gal fusion protein exhibiting β-galactosidase activity. Following in situ staining (Constance 1995), infected cells turn blue due to an enzyme mediated reaction with the chromogenic substrate X-gal (5-bromo-4-chloro-3-indolyl β-D-galactopyranoside), and uninfected cells remain colorless because of the absence of enzyme.

To determine the effect of β-CD on the expression of an IE ICP27-β-galactosidase fusion gene, β-galactosidase reporter gene activity of d27-lacZ1 was monitored by in situ staining of infected Vero cells at 8-12 hours post-infection (PI).

In the first experiment, infected cells in two wells (numbers 1 and 4) of a six-well plate did not receive any treatment (control wells). In two other wells (numbers 2 and 5), infected cells were treated with β-CD during the post-infection period only, whereas in the third set of wells (numbers 3 and 6), infected cells were treated with β-CD both during pre- and post-infection periods.

In a second experiment, the effects of β-CD and acyclovir on the expression of IE ICP27 promoter driven β-galactosidase (HSV-1 vector) were monitored. In two wells of a ‘control’ six-well plate, infected Vero cells were treated with acyclovir both during pre- and post-infection periods. In one well of a ‘test plate’ (a second six-well plate), cells were infected with vector but no treatment performed (positive control) (well number 1). In two other wells (numbers 2 and 5) of the ‘test plate’, infected cells were treated with β-CD only during post-infection period, and in two additional wells (numbers 3 and 6), infected cells were treated with β-CD both during pre- and post-infection periods. The final well (number 4) of the ‘test plate’ contained mock infected cells (negative control).

Results

In the first experiment, in situ staining performed at 8-12 hours post-infection determined the effect of β-CD on the expression of IE promoter driven ICP27-β-galactosidase fusion gene. A noticeable difference was revealed in the expression of reporter gene between duplicate wells number 2 and 5 representing infected cells treated with β-CD only after the vector infection and wells number 3 and 6 representing infected cells treated with β-CD both before and after the vector infection (i.e., β-CD was present throughout the pre-infection and post-infection periods) (plate not shown). This result indicates that β-CD acts early in the infection process, perhaps by limiting virus entry (penetration) or early post-entry events.

In the second experiment that tested the effects of β-CD alone and acyclovir on the expression of IE ICP27 driven β-galactosidase, it was found that treatment with acyclovir in a pre-plus post-infection fashion (‘control plate’) has no effect on the expression of ICP 27 promoter driven reporter gene (which was as expected because acyclovir affects HSV infection at later events). On the contrary, treatment with β-CD (‘test plate’, wells number 3 and 6) almost totally inhibited the expression of the reporter gene. As with the first experiment, this data indicates that β-CD acts early in the infection cycle (up to the expression of IE genes, which includes ICP 27), and hence its mode of action is distinct from acyclovir.

### EXAMPLE 4

Effect of β-CD on HSV Entry

Studies with d27-lacZ1 indicated that β-CD appears to block a step in virus replication cycle prior to, or at, the stage of IE gene expression. The mechanism by which β-CD interferes with HSV growth was further investigated by the following experiment.

Materials and Methods

The effects of β-CD on the yield of intracellular HSV DNA extracted from the Vero cells infected with the recombinant virus (d27-lacZ1) were investigated. Vero cells were grown in six-well plates or T75 flasks and the used media from 24 hours old confluent cultures were replaced with MEM diluted to 1× either with water (control cells) or stock β-CD solution diluted to final concentration of 7.2 mg/ml (test cells). This pre-infection treatment lasted for two hours, after which these media were aspirated. Cells were infected with d27-lacZ1 (β-gal HSV) at MOI of 0.01 or 0.1 for 2 hours. Infections were terminated by aspirating the virus dilution, exposing monolayers to pH 3 citrate buffer momentarily (seconds), and immediately washing mono-layers with PBS for two times. Cells were resuspended in respective MEM media for post-infection treatment. Control cells received 1× MEM, whereas test cells received 1× MEM containing 5.4 mg/ml of β-CD. Following 1 hour of this treatment, different groups of cells were washed with PBS, harvested with trypsin, washed twice and finally resuspended in PBS. RNAase A (DNAase free) was added, and whole cell genomic DNA was prepared using Qiagen DNA Mini Kit (mammalian cells DNA extraction) protocol. All DNA preparations were eluted in 400 μl and analyzed by agarose gel electrophoresis and OD determination at A₂₆₀ and A₂₈₀.

Monolayers treated under identical conditions were incubated for an additional 8-12 hours and subjected to in situ staining for β-galactosidase activity. β-CD inhibition of IE-promoter driven expression of β-galactosidase was confirmed in test cells (as described above).

PCR amplifications were performed on equal amounts of template DNA to compare quantities of viral DNA in a semi-quantitative fashion using sets of PCR primers described below. These experiments were conducted to compare the amount of viral DNA isolated from the control versus β-CD treated infected Vero cells. New sets of primers were designed and PCR conditions optimized both for the viral and cellular DNA in order to optimize results. All the results presented in this study were obtained using a second set of PCR primers designed for monkey β-actin gene (this study) and the published gpB primer set for HSV (Ramrakrishnan, 1994). Sequence of the monkey β-actin gene has been published and was obtained from NCBI (GenBank accession #AB004047). Sequences of the PCR primers for the monkey β-actin are 5′-TGC TGT CCC TGT ACG CCT CT-3′ (SEQ ID NO:1) for the top or forward primer (referred to as B-actin, 5′), and 5′-AGT CCA GGG CGA CAT AGC AC-3′ (SEQ ID NO:2) for the bottom or reverse primer (referred to as B-actin, 3′). For HSV DNA detection, PCR primers set consist of the sequence within glycoprotein B (gpB) gene of HSV-1 as published (Ramakrishnan, 1994). The gpB primer set consists of a “5′ primer”, 5′-ATT CTC CTC CGA CGC CAT ATC CAC CAC CTT-3′ (SEQ ID NO:3) (referred to as gB5′); and a “3′ primer”, 5′-AGA AAG CCC CCA TTG GCC AGG TAG T-3′ (SEQ ID NO:4) (referred to as gB-³′wt).

All PCR reactions were performed using “HotStarTaq Master Mix Kit” (Qiagen). Final volumes of all reactions were set at 50 μl. After mixing contents, PCR tubes were placed on Gene Amp PCR System 2400 (Perkin Elmer). The program used to amplify consisted of holding reactions 15 minutes at 95° C. to activate the enzyme (per manufacturer's recommendation), followed by 30 cycles consisting of denaturation at 94° C. for 45 seconds, annealing at 60° C. for 60 seconds, and extension at 72° C. for 90 seconds. This was followed by a final extension of 10 minutes at 72° C., and final hold at 4° C. until the tubes were retrieved.

5 μl each of the PCR reactions were loaded on 1.5% agarose gel. Lanes 1 and 12 of the gel were loaded with 0.25 μg of 100 bp ladder (molecular weight marker, from New England Biolabs). Lanes 2 to 6 of the gel were loaded with PCR reactions using 20 pmole each of the HSV specific gpB primers (see above), whereas lanes 7 to 11 of the gel were loaded with PCR reactions using 20 pmole each of the Monkey β-actin gene primers (see above). Lanes 2 and 7 each had 1× of DNA from uninfected cells. Lanes 3 and 8 each had 1× of DNA from cells infected with HSV-1 d-27 virus at MOI of 0.01 and treated with β-CD. Lanes 4 and 9 each had 1× of DNA from test cells infected with HSV-1 d-27 virus at MOI of 0.1 and treated with β-CD. Lanes 5 and 10 each had 1× of DNA from control cells infected with HSV-1 d-27 virus at MOI of 0.01 which did not receive treatment. Lanes 6 and 11 each had 1× of DNA from control cells infected with HSV-1 d-27 virus at MOI of 0.1 which did not receive treatment.

The linear range for the HSV specific PCR products observed in lanes 3 and 5 of the first gel was then studied by successively raising the quantities of two template DNA in new PCR reactions and studying effects on the yield of PCR products (quantities compared in a semi-quantitative fashion). 5 μl of PCR reactions were loaded onto a second 1.5% agarose gel. Lanes 1 and 14 of the gel were loaded with 0.25 μg of 100-bp ladder (molecular weight marker). Lanes 2 to 8 were loaded with PCR reactions using 20 pmole each of the first set of HSV gpB primers. Lanes 9 to 13 were loaded with PCR reactions using 20 pmole each of the second set of monkey β-actin gene primers. Lanes 2 and 9 had 2× of DNA from uninfected cells. Lanes 3, 4, and 5 had 1×, 2×, and 3×, respectively, concentrations of DNA from cells infected with HSV-1 d-27 virus at MOI of 0.01 and treated with β-CD. Lanes 6, 7, and 8 each had 1×, 2×, and 3×, respectively, concentrations of DNA from control cells infected with HSV-1 d-27 virus at MOI of 0.01 and no treatment. Lanes 10 and 11 each had 1× and 3× concentrations of DNA from cells infected with HSV-1 d-27 virus at MOI of 0.01 and treated with β-CD, respectively. Lanes 12 and 13 had 1× and 3× concentrations of DNA from control cells infected with HSV-1 d-27 virus at MOI of 0.01 and no treatment, respectively.

The linear range for the HSV specific PCR products observed in lanes 4 and 6 of the first gel was also studied by successively raising the quantities of the two template DNA in new PCR reactions and studying the effects on the yield of PCR products (quantities compared in a semi-quantitative fashion). 5 μl of PCR reactions were loaded onto a third 1.5% agarose gel. Lanes 1 and 6 of the gel were loaded with 0.25 μg of a 100-bp ladder (molecular weight marker). Lanes 2 to 5 were loaded with PCR reactions using 20 pmole each of the first set of HSV gpB primers. Lanes 2 and 3 had 1× and 2.5×, concentrations of DNA from test cells infected with HSV-1 d-27 virus at MOI of 0.1 and treated with β-CD, respectively. Lanes 4 and 5 were loaded with 1× and 2.5× concentrations of DNA from control cells infected with HSV-1 d-27 virus at MOI of 0.1 that received no treatment, respectively.

Results

Although the amount of viral DNA extracted from cells infected with relatively high titer virus (MOI of 0.1) yielded PCR amplifications that were entering plateau phase the amount of viral DNA (‘A’ and ‘D’ DNA; see below for description) extracted from cells infected with lower titer virus (MOI of 0.01) yielded PCR amplifications which were still in log phase, thereby allowing comparison of the amount of viral DNA in between control (‘A’ DNA) and test cells (‘D’ DNA) in these experiments. Results were consistent in repeated experiments. No significant differences between the amount of viral DNA extracted from control and β-CD treated cells were observed.

These data indicate that the anti-viral effect of β-CD is probably not due to inhibition of virus entry. Thus, the antiviral effect of β-CD most likely is exerted at some post entry step(s), i.e., before or at the level of IE gene expression (since β-CD inhibits expression of IE gene promoter (α-27 gene) driven β-galactosidase).

The three gels described above (gels not shown) present data regarding the effect of β-CD on the yield of intracellular virus DNA following HSV infection of Vera cells, which is taken as an evidence of whether β-CD acts by inhibiting the entry of the HSV virus into the host cell. There was no significant quantitative difference in the quantities of virus-specific PCR products amplified from equal amounts of template DNA extracted from control and β-CD treated cells infected with the virus at respective MOI. Thus, the previously demonstrated inhibitory effect β-CD on IE gene expression of HSV is probably not acting at the level of virus entry into the host cell. β-CD may have a specific mode of action by interfering at some step/s following virus entry, up to the IE viral gene expression.

In the first gel, a virus-specific PCR product was not detected and hence the viral DNA was absent in the uninfected cells (lane 2) where the presence of template host cell DNA was demonstrated by amplification of the cellular gene (lane 7). There was no significant difference between the quantities of virus-specific PCR products amplified from the template DNA extracted from control and β-CD treated cells infected with virus at respective MOI (lane 3 vs. 5 and lane 4 vs. 6).

In the second gel, a virus-specific PCR product from the uninfected cells (lane 2) was absent, where the presence of template DNA was demonstrated by amplification of the cellular gene (lane 9). There was a subsequent increase in the amount of virus specific PCR products proportional to the amount of template DNA, both for the test DNA (lanes 3 to 5) and the control DNA (lanes 6 to 8), thereby establishing a linear range. For the cellular gene, all amounts of template DNA allowed the progression of PCR to the plateau phase, and hence no significant increases were observed in the amount of P-actin gene specific PCR products produced with respect to the amounts of template DNA added (lanes 9 to 13).

The third gel showed an increase in the amount of virus specific PCR product that somewhat correlated with an increase in the amount of template DNA, both for the test DNA (lane 3 vs. 2) and the control DNA (lane 5 vs. 4). However, this increase did not appear proportional, probably because the reactions were entering plateau phase.

Discussion

β-CD alone exhibits potent activity against HSV-1 (KOS1.1) and HSV-2 (MS) to a MOI of 2 (FIG. 1). At the final concentration of 7.2 mg/ml of β-CD, this activity was found comparable to acyclovir in the range of 200 to 400 μg/ml. The experiments herein (Examples 3 and 4) demonstrate that the β-CD acts at an early stage of the replication of HSV, most probably at a post-entry step, such as uncoating, transport of the viral genome to the nucleus, or IE gene expression itself. This is different than the reported mode of action of acyclovir, which acts at the DNA replication step of the virus infection cycle. This distinct mode of action holds the potential for the additive benefit of β-CD and acyclovir in anti-viral therapies. In fact, preliminary studies regarding the possible synergistic effect of acyclovir and β-CD demonstrated more than additive effects of the combined formulation.

Another important observation is the finding that β-CD is effective against acyclovir-resistant HSV (FIG. 2). This indicates not only a distinct mode of action exerted by β-CD that has been verified by further experiments (Examples 3 and 4), but also it has significant implications in the prevention of and/or therapeutics for problematic HSV. In addition to HSV, these studies can be extended to the anti-viral effect of cyclodextrins, such as β-CD, against drug resistant strains of other important viral pathogens.

Thus, topical and other formulations containing β-CD alone or in combination with other anti-HSV compounds, e.g., acyclovir and other nucleoside analogs, are very effective therapies against herpes viruses, in particular, HSV-1 and HSV-2.

### EXAMPLE 5

Antiviral Activity of Methyl-β-CD and β-CD against Vaccinia Virus

In vitro anti-viral assays using beta-cyclodextrin (BCD) and methyl-beta-cyclodextrin (MBCD) demonstrated the anti-vaccinia virus activity of these compounds.

In a six-well plate, Vero cells were grown in monolayers in MEM and infected with vaccinia Western Reserve strain (WR) (VR-119) up to a dose of 10^{3.5} TCID₅₀ for about two hours. Cells were treated before (approximately three hours) and following infection with MBCD at 4 to 4.5 mg/ml. Cells were treated with MBCD for 48 hours, fixed and stained with 0.5% crystal violet plus 35% methanol soluation and photographed (photograph not shown).

Well 1 of the six-well plate contained a monolayer of uninfected Vero cells. Well 4 contained uninfected Vero cells with MBCD treatment. Wells 2 and 5 contained vaccinia infected untreated cells. Wells 3 and 6 contained infected and MBCD treated cells. The MBCD treated wells showed much fewer and smaller areas of cytopathic effect as compared to the untreated wells. This level of MBCD is below the level of any significant MBCD cell killing. Treatment with β-CD (at 7.2 mg/ml) showed similar levels to MBCD of anti-vaccinia virus activity (data not shown).

Using the Live/Dead® Viability/Cytoxicity Assay Kit (L-3224) (Molecular Probes, Eugene, Oreg.), cytoxicity assays for BCD and MBCD were conducted on Vero cells to ensure the anti-vaccinia activity was not due to cell cytotoxicity. This assay is a two-color fluorescence cell viability assay based on simultaneous determination of live and dead cells utilizing a flow cytometer (FACSalibur, BD Biosciences, San Jose, Calif.). Live cells were distinguished by the presence of intracellular esterase activity, determined by the enzymatic conversion of calcein-AM (nonfluorescent) to calcein (green fluorescence). Ethidium homodimer-1 (EthD-1) enters cells with damaged cell membranes and binds to nucleic acids producing red fluorescence to identify dead cells. EthD-1 is excluded from cells with intact plasma membranes (live cells). The background fluorescence of this assay is inherently low, because the dyes are virtually non-fluorescent before interacting with cells.

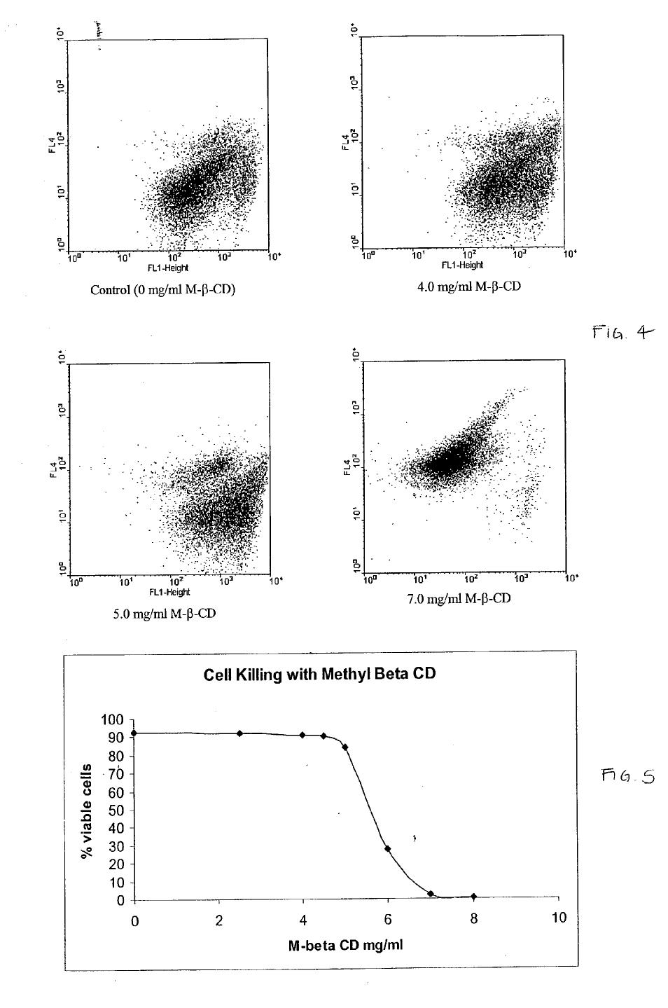

Briefly, Vero cells were cultured in 5% FBS-MEM in six-well plates. Once monolayers were formed, MEM containing either BCD (Sigma product number C4805) or MBCD (Sigma product number C4555) was added at the final concentrations shown in FIG. 4 (e.g., at 0, 4.0, 5.0, 7.0 mg/ml etc.). After 48 hours, the medium was removed, cells trypsinized and resuspended in MEM plus 1 μM calcein and 4 μM EthD-1 for flow cytometry studies. Control cells were killed with methanol to establish live/dead scattergram patterns.

The degree of separation of live and dead cell populations was determined by comparing the control scattergram (0 mg/ml MBCD) and the highest treatment level shown (7.0 mg/ml MBCD). As the concentration of MBCD increased, fewer live cells (green fluorescence) were seen in the lower right hand corner of each scattergram, and more dead cells (red fluorescence) were seen in the upper-central portion of the scattergram. These data were used to generate the cell killing graph shown in FIG. 5 for Vero cells exposed to MBCD for 48 hours. Extrapolating these data indicate the CC₅₀ at 48 hours for Vero cells is approximately 5.5 mg/ml of MBCD. This is significant because MBCD is reportedly one of the more cytotoxic cyclodextrin compounds, and yet has a very abrupt threshold before dose related cell killing is observed. Anti-vaccinia virus activity of MBCD was observed in the flat portion of this curve (<4.5 mg/ml), well below the dosage at which Vero cell killing occurs.

For BCD, solubility becomes a limiting issue at greater than 8 mg/ml, however, <15% cytotoxicity was observed after exposing Vero cells to the maximum concentration of BCD for 48 hours. Other cyclodextrins may have an even better selectivity index, thereby demonstrating that these compounds have great potential as anti-vaccinia virus agents.

### EXAMPLE 6

Cyclodextrins Extract Cholesterol from Membrane Domains

In six-well plates, Vero cells were infected with HSV-1 KOS 1.1 at an MOI of 2 and 0.002 were cultured. The effects of MBCD (4 mg/ml for 48 hours for MOI of 2, and 4-5 days for MOI of 0.002; in wells 2 and 5 of each plate) versus MBCD/cholesterol complex (wells 3 and 6) were examined.

The MBCD/cholesterol complex is a saturated solution made by the following procedure: 2 grams of MBCD were added to 40 ml of water, heated and kept at 80° C. A solution containing 60 mg of cholesterol dissolved in 20 ml of 2-propanol was then added very slowly to complete solubility, and the complex was evaporated. The resulting powdered complex had 30 mg cholesterol per gram of MBCD. A stock solution with 10 mg/ml MBCD (and hence 0.3 mg/ml of cholesterol) was used to obtain final concentrations of 4, 6, or 8 mg/ml with respect to MBCD in these experiments.

Well 1 contained untreated infected cells, while well 4 contained uninfected control cells. All wells were stained for adherent cells 48 hours post-infection (data not shown).

Studies on the antiviral mechanism of action of BCD using HSV-1 and Vero cells have been conducted (see Example 3). Here, MBCD treatment (4 mg/ml) alone provided potent anti-herpes protection, while MBCD complexed to cholesterol provided no protection from virus infection. MBCD was used because of its slightly higher cytotoxicity to Vero cells compared to β-CD, better solubility and lack of crystal formation. Cyclodextrin (CD), e.g., β-cyclodextrin and methyl β-cyclodextrin, complexes to cholesterol in a 1:1 fashion and eliminates the cholesterol depleting effects of CD.

The data shows that the cyclodextrins extract cholesterol from membrane domains that are essential for the productive infection and proliferation of various viruses, including Herpes. It is known that the cyclodextrins bind free cholesterol. Saturating the binding sites with cholesterol abolishes the anti-herpes activity of methyl-β-cyclodextrin.

### EXAMPLE 7

Exemplary Procedures for Determining Antiviral Efficacy and Toxicity

The antiviral activity of cyclodextrins (e.g., α-cyclodextrin, β-cyclodextrin and γ-cyclodextrin) and derivatives thereof (e.g., hydroxypropyl α-cyclodextrin, hydroxypropyl β-cyclodextrin and hydroxypropyl γ-cyclodextrin) can be examined using assays and techniques well-known to the art, such as described hereinbelow. For an experimental drug, in vitro studies such as described herein can be conducted to determine the EC50 (effective concentration 50), which is the concentration required to inhibit viral replication by 50%; the CC50 (cytotoxic concentration 50), which is the concentration required to inhibit 50% stationary cells to take up neutral red; the IC50 (inhibitory concentration 50), which is the concentration required to inhibit cell growth by 50%; as well as the SI (selective index), the ratio of CC50/EC50. The 50% and 90% effective antiviral concentrations (EC₅₀, EC₉₀) and the 50% cytotoxic concentrations (CC₅₀) can also be calculated and used to generate Selectivity Indexes (CC₅₀/EC₅₀). An S.I. of 10 or greater is considered to be a selective antiviral effect.

A. Preparation of Human Foreskin Fibroblast Cells

Newborn human foreskins were obtained as soon as possible after circumcisions were performed and placed in minimal essential medium (MEM) containing vancomycin, fungizone, penicillin, and gentamycin, at the usual concentrations, for four hours. The medium was then removed, the foreskin minced into small pieces and washed repeatedly until red cells were no longer present. The tissue was then trypsinized using trypsin at 0.25% with continuous stirring for 15 minutes at 37° C. in a CO₂ incubator. At the end of each 15-minute period the tissue was allowed to settle to the bottom of the flask. The supernatant containing cells was poured through sterile cheesecloth into a flask containing MEM and 10% fetal bovine serum. The flask containing the medium was kept on ice throughout the trypsinizing procedure. After each addition of cells, the cheesecloth was washed with a small amount of MEM containing serum. Fresh trypsin was added each time to the foreskin pieces and the procedure repeated until no more cells became available. The cell-containing medium was then centrifuged at 1000 RPM at 4° C. for ten minutes. The supernatant liquid was discarded and the cells resuspended in a small amount of MEM with 10% FBS. The cells were then placed in an appropriate number of 25 cm² tissue culture flasks. As cells became confluent and needed trypsinization, they were expanded into larger flasks. The cells were kept on vancomycin and fungizone to passage four.

B. Cytopathic Effect Inhibition Assay—HSV, HCMV, VZV

Low passage human foreskin fibroblast cells were seeded into 96 well tissue culture plates 24 hours prior to use at a cell concentration of 2.5×10⁵ cells per ml in 0.1 ml of minimal essential medium (MEM) supplemented with 10% fetal bovine serum (FBS). The cells were then incubated for 24 hours at 37° C. in a CO₂ incubator. After incubation, the medium was removed and 125 μl of experimental drug was added in triplicate wells. The drug in the first row of wells was then diluted serially 1:5 throughout the remaining wells by transferring 25 μl using the Cetus Liquid Handling Machine. After dilution of drug, 100 μl of the appropriate virus concentration was added to each well, excluding cell control wells, which received 100 μl of MEM. For HSV-1 and HSV-2 assays, the virus concentration utilized was 1000 PFU's per well. For CMV and VZV assays, the virus concentration added was 2500 PFU per well. The plates were then incubated at 37° C. in a CO₂ incubator for three days for HSV-1 and HSV-2, 10 days for VZV, or 14 days for CMV. After the incubation period, media was aspirated and the cells stained with a 0.1% crystal violet solution for four hours. The stain was then removed and the plates rinsed using tap water until all excess stain was removed. The plates were allowed to dry for 24 hours and then read on a BioTek Plate Reader at 620 nm.

C. Plaque Reduction Assay for HSV-1 and HSV-2 Using Semi-Solid Overlay

Two days prior to use, HFF cells were plated into six well plates and incubated at 37° C. with 5% CO₂ and 90% humidity. On the date of assay, the drug to be tested was made up at twice the desired concentration in 2% MEM and then serially diluted 1:5 in 2% MEM using six concentrations of drug. The initial starting concentration was approximately 200 μg/ml down to 0.06 μg/ml. The virus was diluted in MEM containing 10% FBS to a desired concentration to give 20-30 plaques per well. The media was then aspirated from the wells and 0.2 ml virus was added to each well in duplicate with 0.2 ml of media added to drug toxicity wells. The plates were then incubated for one hour with shaking every fifteen minutes. After the incubation period, an equal amount of 1% agarose was added to an equal volume of each drug dilution. This will give final drug concentrations beginning with 100 μg/ml and ending with 0.03 μg/ml and a final agarose overlay concentration of 0.5%. The drug agarose mixture was applied to each well in 2 ml volume and the plates then incubated for three days, after which the cells were stained with a 1.5% solution of neutral red. At the end of 4-5 hour incubation period, the stain was aspirated, and plaques counted using a stereomicroscope at 10× magnification.

D. VZV Plaque Reduction Assay—Semi-Solid Overlay

The procedure was conducted essentially the same as for the HSV plaque assay described above, with the following two exceptions:

1. After addition of the drug, the plates were incubated for ten days.

2. On days three and six, an additional 1 ml overlay with equal amounts of 2× MEM and 1% agarose was added.

E. CMV Plaque Assay—Semi-Solid Overlay

The procedure was conducted essentially the same as for the HSV plaque assay, with the following changes. The agarose used for both the initial overlay and the two subsequent overlays was 0.8% rather than 1%. The assay was incubated for 14 days with the additional 1 ml overlays applied on days four and eight.

F. Plaque Reduction Assays Using Liquid Medium Overlay

The procedure for the liquid overlay plaque assay was similar to that using the agarose overlay. The procedure for adding virus was the same as for the regular plaque assay. Solutions of the experimental drugs were made up in MEM with 2% FBS. The drugs were not made up at 2× concentration as in the previous assays, but at the desired concentration. For HSV-1 and HSV-2 assays, an antibody preparation obtained from Baxter Health Care Corporation was diluted 1:500 and added to the media that the drug was diluted in. For CMV and VZV, no antibody in the overlay was utilized. For the CMV assay, additional medium without new drug was added on day four and allowed to incubate for a total of 8 days. For VZV, additional media was added on day five and incubated for a total of 10 days. At the end of the incubation period for all of the assays, 1 ml of crystal violet was added to each well. The cells were stained 10 minutes, washed with PD, and plaques then enumerated using a stereomicroscope.

G. Screening and Confirmation Assays for EBV

Virus: Two prototypes of infectious EBV are available. One is exemplified by the virus derived from supernatant fluids of the P3HR-1 cell line, which produces nontransforming virus that causes the production of early antigen (EA) after primary infection or superinfection of B cell lines. The other prototype is exemplified by the B-95-8 virus, which immortalized cord blood lymphocytes and induced tumors in marmosets. It does not, however, induce an abortive productive infection even in cell lines harboring EBV genome copies. The virus used in the assays described herein was P3HR-1.

Cell Lines: Daudi cells are a transformed cell like that produce a low level of EBV (152 EBV genome copies/cell). It spontaneously expresses EBV EA in 0.25%-0.5% of the cells. These cell lines respond to superinfection by FBV by expressing EA(D), EA(R), and viral capsid antigen (VCA). This cell line was maintained in RPMI-1640 medium supplemented by 10% FCS, L-glutamine and 100 ug/ml gentamicin. The cultures were fed twice weekly and the cell concentration adjusted to 3×10⁵/ml. The cells were kept at 37° C. in an humidified atmosphere with 5% CO₂.

Immunofluorescence Assays with Monoclonal Antibodies: Cells were infected with the P3HR-1 strain of EBV and the drugs to be tested were added after adsorption (45 minutes at 37° C.) and washing of the cell cultures.

The cultures were incubated for two days in complete medium to allow viral gene expression. Following the 48 hour incubation period, the number of cells of each sample was counted and smears were made. Monoclonal antibodies to the different EA components and VCA were then added to the cells incubated and washed. This was followed by a flourescein conjugated rabbit anti-mouse Ig antibody. The number of fluorescence positive cells in the smears were counted. The total number of cells in the cultures positive for EA or VCA were then calculated and compared.

H. Cell Proliferation Assay—Toxicity

Twenty-four hours prior to assay, HFF cell were seeded in 6-well plates at a concentration of 2.5×10⁴ cells per well in MEM containing 10% FBS. On the day of the assay, drugs were diluted serially in MEM containing 10% FBS at increments of 1:5 covering a range from 100 μg/ml to 0.03 μg/ml. For drugs that have to be solubilized in DMSO, control wells received MEM containing 10% DMSO. The media from the wells was then aspirated and 2 ml of each drug concentration was then added to each well. The cells were then incubated in a CO₂ incubator at 37° C. for 72 hours. At the end of this time, the media-drug solution was removed and the cells washed. One ml of 0.25% trypsin was added to each well and incubated until the cells started to come off of the plate. The cell-media mixture was then pipetted up and down vigorously to break up the cell suspensions, and 0.2 ml of the mixture was added to 9.8 ml of Isoton III and counted using a Coulter Counter. Each sample was counted three times with two replicate wells per sample.

I. Neutral Red Uptake Assay—Toxicity

Twenty-four hours prior to the assay, HFF cells were plated into 96 well plates at a concentration of 2.5×10⁴ cells per well. After 24 hours, the media was aspirated and 125 μl of drug was added to the first row of wells and then diluted serially 1:5 using the Cetus Liquid Handling System in a manner similar to that used in the CPE assay.

After drug addition, the plates were incubated for seven days in a CO₂ incubator at 37° C. At this time the media/drug was aspirated and 200 μl/well of 0.01% neutral red in DPBS was added. This was incubated in the CO₂ incubator for one hour. The dye was then aspirated, and the cells were washed using a Nunc Plate Washer. After removing the DPBS wash, 200 μg/well of 50% ETOH/1% glacial acetic acid (in H₂O) was added. The plates were rotated for 15 minutes and the optical densities were read at 540 nm on a plate reader.

Results

Procedures similar to those described above were used to evaluate the anti-viral activity of α-cyclodextrin, β-cyclodextrin, γ-cyclodextrin, hydroxypropyl α-cyclodextrin, hydroxypropyl β-cyclodextrin and hydroxypropyl γ-cyclodextrin. Data collected from the in vitro screening are summarized in Table 1. The data depicted in Table 1 were generated, in general, using 1000-fold less experimental drug than used in Examples 1-6 and 10. In addition, different strains of virus were employed as compared to those in Examples 1-6 and 10.

TABLE 1

| Virus | ACD | BCD | GCD | HPACD | HPBCD | HPGCD | MBCD |
|---|---|---|---|---|---|---|---|
| HSV-1 | 0 | 0 | 0 | 0 | 0 | 0 | 0 |
| HSV-2 | 0 | 0 | 0 | 0 | 0 | 0 | 0 |
| VZV | 0 | 0 | 0 | 0 | 0 | 0 | 0 |
| EBV* | >1.5 | 0 | 0 | 0 | >625.(2.5) | >625.(6.2) | 0 |
| HCMV | 0 | 0 | 0 | 0 | 0 | 0 | 0 |
| MCMV | 0 | 0 | 0 | 0 | 0 | 0 | 0 |
| HHV-6 | 0 | 0 | 0 | 0 | 0 | 0 | 0 |
| HHV-7 | 0 | 0 | 0 | 0 | 0 | 0 | 0 |
| HHV-8** | | | | | | | |
| Flu A | 0 | 0 | 0 | 0 | 0 | 0 | 0 |
| Flu B | 0 | 0 | 0 | 0 | 0 | 0 | 0 |
| RSV | 0 | 0 | 0 | 0 | 0 | 0 | 0 |
| PIV | 0 | 0 | 0 | 0 | 0 | 0 | 0 |
| MV | 0 | 0 | 0 | 0 | 0 | 0 | 0 |
| HRV | 0 | 0 | 0 | 0 | 0 | 0 | 0 |
| Ad | 0 | 0 | 0 | 0 | 0 | 0 | 0 |
| Resp | 0 | 0 | 0 | 0 | 0 | 0 | 0 |
| syncytial | | | | | | | |
| Rhino | 0 | 0 | 0 | 0 | 0 | 0 | 0 |
| Vaccinia | 0 | 0 | 0 | 0 | 0 | 0 | 0 |
| Cowpox | 0 | 0 | 0 | 0 | 0 | 0 | 0 |
| Measles | 0 | 0 | 0 | 0 | 0 | 0 | 0 |
| VEE | 0 | 0 | 0 | 0 | 0 | 0 | 0 |
| Punta | 0 | 0 | 0 | 0 | 0 | 0 | 0 |
| Toro | | | | | | | |
| Pichinde | 0 | 0 | 0 | 0 | 0 | 0 | 0 |
| Yellow | 0 | 0 | 0 | 0 | 0 | 0 | 0 |
| fever | | | | | | | |
| West | 0 | possible | 0 | 0 | 0 | 0 | 0 (?) |
| Nile | | | | | | | |

(SI = CC50/EC50, i.e., Tox/AntiViral; % control)
*For EBV, values in parenthesis were calculated by DNA hybridization.
**No data for HHV-8 provided.

### EXAMPLE 8

The anti-hepatitis C activity of cyclodextrins (e.g., α-cyclodextrin, β-cyclodextrin and γ-cyclodextrin) and derivatives thereof (e.g., hydroxypropyl α-cyclodextrin, hydroxypropyl β-cyclodextrin and hydroxypropyl γ-cyclodextrin) can be examined using assays and techniques well-known to the art, such as described hereinbelow.

Primary In Vitro Anti-HCV Assay

The antiviral activity of test compounds were assayed in the stably HCV RNA-replicating cell line. AVA5, derived by transfection of the human hepatoblastoma cell line, Huh⁷ (Blight et al., 2000). Experimental drugs were added to dividing cultures once daily for three days. Media was changed with each addition of compound. Cultures generally started the assays at 50% confluence and reached confluence during the last day of treatment. HCV RNA and cellular β-actin RNA levels were assessed 24 hours after the last dose of compound using dot blot hybridization. Assays were conducted using a single dose of test compound (in triplicate cultures). A total of 6 untreated control cultures, and triplicate cultures treated with 10 IU/ml of α-interferon (the approximate EC₉₀ with no cytotoxicity) and 100 μM of ribavirin (the approximate CC₉₀ with no antiviral activity) served as the positive antiviral and toxicity controls.

Both HCV and β-actin RNA levels in the treated cultures were expressed as a percentage of the mean levels of RNA detected in untreated cultures. β-actin RNA levels were used both as a measure of toxicity, and to normalize the amount of cellular RNA in each sample. A level of 30% or less HCV RNA (relative to control cultures) was considered to be a positive antiviral effect, and a level of 50% or less β-actin RNA (relative to control cultures) is considered to be a cytotoxic effect.

Secondary In Vitro Anti-HCV Assay

Dividing cultures of AVAB cells were treated once daily for three days (media was changed with each addition of compound) with 4 concentrations of test compounds (3 cultures per concentration). A total of 6 untreated control cultures, and triplicate cultures treated with 10, 3, and 1 IU/ml α-interferon (active antiviral with no cytotoxicity), and 100, 10, and 1 μM ribavir (no antiviral activity and cytotoxic) served as controls. HCV RNA and cellular β-actin FNA levels were assessed 24 hours after the last dose of compound using dot blot hybridization. β-actin RNA levels were used to normalize the amount of cellular RNA in each sample.

Toxicity analyses were performed on separate plates from those used for the antiviral assays. Cells for the toxicity analyses were cultured and treated with test compounds with the same schedule and under identical culture conditions used for the antiviral evaluations. Each compound was tested at 4 concentrations, each in triplicate cultures. Uptake of neutral red dye was used to determine the relative level of toxicity 24 hours following the last treatment. The absorbance of internalized dye at 50 nM (A₅₁₀) was used for the quantitative analysis. Values in test cultures were compared to 9 cultures of untreated cells maintained on the same plate as the test cultures.

Results

Assays similar to those described above were conducted.

The S.I. for HCV was 0.72 for α-CD, 1.13 for β-CD, 1.04 for δ-CD, 0.97 for HPACD, 1.26 for HPBCD, 1.39 for HPGCD, and 1.69 for MBCD.

### EXAMPLE 9

In Vivo Anti-Herpesvirus Activity of Beta-Cyclodextrin

Herpes viruses cause a range of acute and chronic illnesses including oral and labial herpes, infectious mononucleosis, herpetic whitlow, herpes zoster, encephalitis and fatal infections of neonates and immunocompromised patients.

Genital herpes is a sexually transmitted disease caused by HSV-2 infection (in 95% of the cases) or HSV-1 infection (in 5% of cases). HSV-2 causes a persistent latent infection leading to recurrent genital lesions facilitating spread to unprotected sexual partners or newborns at the time of delivery. It causes significant morbidity and may cause life threatening illness in immunocompromised individuals. HSV-2 infection also increases the risk of HIV transmission. Despite antiviral therapy and public health education, over the last two decades the incidence of genital herpes has increased significantly.

While a number of antiviral agents show activity against these viruses, none can cure the infection once latency is established and herpes virus resistance to standard therapy exists. Acyclovir (ACV) treatment is effective at reducing the length and severity of herpes eruptions, but does not eliminate the potential for transmission or latent infections. Furthermore, acyclovir resistance may develop. The in vitro anti-herpes activity of β-cyclodextrin (BCD), a cyclic oligosaccharide, against HSV-1 and HSV-2 is disclosed herein. (See also Khan et al., submitted.) β-cyclodextrin derivatives have also been shown to have potent anti-HIV activity (Liao et al., 2001) and anti-CMV activity (Leydet et al., 1998).

Cyclodextrins are cyclic oligosaccharides with either six (α), seven (β), or eight (γ) sugar units. β-cyclodextrin is commonly used as a drug carrier because it has aqueous solubility, can bind lipophilic agents in its central core, and is relatively non-toxic. As disclosed herein, potent anti-HSV-1 and anti-HSV-2 activity, including effectiveness against acyclovir-resistant HSV, if β-cyclodextrin has been shown using Vero cells in vitro. In addition, cytotoxicity studies establishing concentrations of BCD that exhibit high anti-herpes activity and low toxicity for cultured cells have been performed. The antiviral activity of β-cyclodextrin and its derivatives have been shown to be possibly linked to an efflux of cholesterol from infected cells treated in culture.

The in vivo activity of β-cyclodextrin against HSV-2 can be studied in an animal model of herpes virus transmission, e.g., a vaginal transmission model. In addition, the mechanism of anti-herpes activity of β-cyclodextrin can be explored, e.g., whether or not β-cyclodextrin anti-herpes activity is mediated by cholesterol efflux from cells can be determined.

To determine if β-cyclodextrin will prevent HSV-2 infection in a mouse vaginal model of genital herpes transmission, and do so by causing cholesterol efflux from the lipid rafts of cell membranes, thus interfering with HSV-2 entry or release from cells.

The ability of β-cyclodextrin to prevent transmission of HSV-2 to mice can be studied using a mouse model of vaginal transmission as previously described (Bourne et al., 1999 and Zeitlin et al., 1997). 48 two-month old female C57B1/6 mice (Harlan Industries, Madison, Wis.) are given long-acting progestin subcutaneously (2.5 mg Depo-Provera; Upjohn, Kalamazoo, Mich.) 7 days prior to virus inoculation to enhance vaginal transmission of HSV-2. On the day of inoculation, mice are anesthetized with 0.025 ml of a solution containing 6.5 mg/ml of sodium pentobarbital by intraperitoneal injection. Equal numbers of animals (see Table 2) are treated by vaginal instillation of 8 mg/ml solution of β-cyclodextrin (BCD) in phosphate buffered saline (PBS), 400 μg/ml acyclovir(ACV) in PBS, ACV/BCD 1:1 complex (400 μg/ml each) in PBS, or PBS alone (control) to compare efficacy of each treatment. Acyclovir has been shown to complex β-cyclodextrin in a 1:1 fashion and the efficacy of ganciclovir (a related nucleoside analog) against cytomegalovirus, another herpes virus, is enhanced by being complexed to β-cyclodextrin (Nicolazzi et al., 2002). Following each test treatment, animals are given either no virus (mock treatment consisting of growth medium minus virus) or 10⁴ PFU (plaque forming units) of HSV-2 strain MS by intravaginal instillation of a 0.015 ml suspension of virus in growth medium. Each mouse is vaginally swabbed on day 2 post-inoculation (PI) and virus stored at −80° C. until assayed by culture in Vero cells for the presence of virus cytopathic effect (CPF). Viral assays will be expressed as CPE scores (indicating the level of anti-viral activity) for each treatment group and compared to control levels using the student t test and p<0.05 level of significance. All mice will be euthanized on day 21 PI for evidence of symptomatic vaginal infection by histological examination of vaginal tissues. Any morbidity will be recorded and compared between treatment and control groups.

TABLE 2

| Drug Treatment | Virus Treatment | Number of Mice |
|---|---|---|
| β-cyclodextrin (BCD) | Mock | 8 |
| PBS only | HSV-2 | 8 |
| BCD | HSV-2 | 8 |
| Acyclovir (ACV) | HSV-2 | 8 |
| ACV/BCD complex | HSV-2 | 8 |
| PBS only | Mock | 8 |

The mechanism of action of β-cyclodextrin anti-viral activity appears to involve cholesterol efflux from cells apparently by binding cholesterol to its central core. Cholesterol is concentrated within segments of the cell membrane known as lipid rafts, which are important sites of viral entry and budding. The anti-HSV-1 activity of methyl-β-cyclodextrin (MBCD) has been eliminated by adding sufficient cholesterol to MBCD prior to in vitro treatment of Vero cells to achieve full occupancy of β-cyclodextrin binding. Cholesterol levels are measured within cells and in the culture medium, prior to and following β-cyclodextrin treatment and HSV-2 infection of Vero cells to determine if cholesterol efflux is coincidental with anti-viral protection. Cholesterol is replaced with β-sitosterol, a plant sterol which is not absorbed by animal cells to determine the specificity of β-cyclodextrin activity regarding cholesterol efflux. Cholesterol efflux is visually documented by staining β-cyclodextrin treated cells with filipin, a fluorescent dye specific for cholesterol and capture images of β-cyclodextrin cells prior to and following β-cyclodextrin treatment. The intensity of fluorescent signal from intracellular cholesterol is compared between cyclodextrin-treated and control cells on a microplate reader to quantify the degree of cholesterol efflux.

### EXAMPLE 10

Formulations of cyclodextrins were studied for antiviral activity against HSV-1 and HSV-2 in Vero cells. Vero cells were exposed pre- and/or post-virus infection to control or treatment culture media containing α-cyclodextrin or β-cyclodextrin (ACD or BCD) and monitored for evidence of viral replication. ACD showed no significant anti-herpes activity. Antiviral activity of BCD was documented by inhibition of cytopathic effects (CPE) caused by HSV-1 and HSV-2 infection, reduction in cell-free and cell-associated virus titers following BCD treatment, and reduction in the ability of a replication deficient HSV-1, d27-lacZ1, to express a β-galactosidase tagged immediate-early (IE) viral gene. BCD at a final concentration of 7-8 mg/ml exhibited anti-herpes effects at a high multiplicity of infection (MOI of >50) for both HSV-1 and HSV-2. BCD reduced cell-free and cell-associated virus at 24 hours post-infection better than acyclovir (ACV) at final concentrations of 200-400 μg/ml, and showed antiviral activity against an ACV-resistant strain of HSV-1. Studies using the d27-lacZ1 virus suggest that unlike ACV, BCD acts at an early stage of virus replication.

Introduction

Herpes viruses are enveloped DNA viruses responsible for a wide spectrum of human disease characterized by lifelong infection with periods of latency and reactivation. Latency aids the virus in avoiding immune surveillance, while reactivation results in recurrent infections and disease with additional opportunities for viral transmission to new hosts. Herpes simplex infection causes orofacial vesicles (HSV-1) or genital lesions (HSV-2). These viruses are also responsible for herpetic keratoconjunctivitis, herpetic whitlow, fatal encephalitis, aseptic meningitis, and an increased risk of acquiring additional sexually transmitted diseases (Mertz, 1997; Taylor et al., 2002). Transmission is typically horizontal, but perinatal transmission can cause serious infection in neonates. Herpes simplex infections are responsible for significant morbidity and mortality in immunocompromised patients. HSV-2 infection may facilitate transmission of the human immunodeficiency virus (HIV) and may increase the rate of HIV replication during both clinical and subclinical HSV-2 infection in co-infected individuals (Aoki, 2001; Schacker, 2001). Antiviral chemotherapy of primary infections using nucleoside analogs can successfully attenuate viral infection and substantially decrease the risk of virus transmission (Dargan, 1998).

Acyclovir (ACV) has been widely used in the treatment of herpes simplex virus infections. It is activated by viral thymidine kinase to a triphosphate form which selectively inhibits viral DNA polymerase, thus preventing viral replication (Elion et al., 1977). ACV treatment does not affect latent virus, but is considered safe and efficacious in both systemic and topical forms. When clinical isolates of HSV are tested in cell culture, the majority are sensitive to ACV. However, ACV-resistant strains of HSV are found in about 1% of isolates from non-immunocompromised patients, and approximately 5% of isolates from immunocompromised individuals (Field, 2001; Shin et al., 2001). Resistance is the result of mutations in viral genes coding for thymidine kinase or DNA polymerase (Schnipper et al., 1980). Because of problems with bioavailability, and resistance to ACV, other anti-herpes agents have been developed. However, some of these agents exhibit significant toxicity (Naesens et al., 2001), which is especially important in small children and pregnant patients. Thus, it is important to search for new drugs to prevent HSV transmission and/or treat HSV infections, particularly in patients resistant to conventional antiviral drug therapies.

Cyclodextrins are water-soluble cyclic oligosaccharides (Loftsson, 1999) and are used as carriers in a number of topical and oral medications, as well as food additives. α-Cyclodextrin (ACD) consists of a six unit oligosaccharide, while β-cyclodextrin (BCD) consists of seven (α-1,4)-linked α-D-glucopyranose units. This arrangement forms a rigid torus-shaped molecule with a polar exterior, and a relatively lipophilic core. Water insoluble agents can be delivered to tissues by incorporation into the central cavity. Recent reports have documented in vitro activity of BCD and its derivatives against HIV and cytomegalovirus (CMV), another herpes virus (Leydet et al., 1998; Liao et al., 2001). The in vitro activity of ACD and BCD formulations was tested against HSV-1 and HSV-2. The in vitro activity of BCD was tested against an ACV-resistant strain of HSV-1.

Materials and Methods

Culture Media, Cells and Viruses

Vero cells were obtained from American Type Culture Collection (ATCC, Rockville, MD) and grown in Modified Eagle's Minimum Essential Medium (MEM; ATCC) containing Earle's balanced salts, non-essential amino acids, 1 mM sodium pyruvate, and 180 mM of sodium bicarbonate adjusted to include 4 mM L-glutamine, 10 percent heat-inactivated fetal bovine serum (FBS; Life Technologies, Gaithersburg, Md.), and an antibiotic-antimycotic solution (Life Technologies) containing 100 units/ml penicillin G, 100 μg/ml streptomycin, and 0.25 μg/ml amphotericin B. For subsequent culturing of cells and antiviral assays, FBS concentration was reduced to 5% (1× MEM-5% FBS).

Sodium pyruvate (Sigma, St. Louis, Mo.), L-glutamine (Mediateci Cellgro®, Herndon, Va.), sodium bicarbonate (Life Technologies), antibiotic-antimycotic solution, and FBS were added to 10× MEM (Eagle's Modified Medium; ICN Biomedicals) to produce 5× MEM-FBS (5 times that of 1× MEM-5% FBS). Different stock solutions of ACV or CD were mixed in a ratio of 4:1 with the 5× medium to formulate the treatment media (1×) for antiviral assays. Water was added to 5× MEM-FBS to serve as control medium.

V27 cells were derived from Vero cells by stable transfection of ICP27 gene of HSV-1 (Rice and Knipe, 1990). These cells were propagated in 1× MEM-5% FBS containing 300 μg/ml of active G418 (Geneticin solution; Life Technologies). Experiments were performed with HSV-1 strain KOS 1.1 (Hughes and Munyon, 1975), HSV-2 strain MS, dlsptk (Coen et al., 1989) (ACV resistant HSV-1), and d27-lacZ1 (Rice and Knipe, 1990) (β-galactosidase expressing recombinant HSV-1). Frozen virus preparations were stored at −80° C.

The d27-lacZ1 virus possesses an intact lacZ gene cloned in-frame with the coding sequence of a partially deleted ICP27 gene (Rice and Knipe, 1990). It expresses an ICP27-β-gal fusion protein that exhibits β-galactosidase activity, which can be used to detect infected cells as previously described (Tebas et al., 1997).

Test Compound Formulations

Cell culture grade α-cyclodextrin (ACD) and β-cyclodextrin (BCD) were obtained from Sigma. Stock formulations containing 9 to 10 mg/ml of either cyclodextrin (CD) were prepared in water and stored at room temperature. 0.5 mg/ml stock solutions of ACV (Acycloguanosine; Sigma) were prepared in water and kept frozen at −20° C. until used. All formulations were passed through 0.2 μm filters prior to use.

CPE Reduction Assays

Effects of CD on viral replication were studied by reduction of CPE on Vero cells grown in six-well plates and used to calculate the IC₅₀ (inhibitory concentration) of BCD. Where indicated, used medium was replaced with 2 ml of medium containing the respective formulations (described herein) to initiate pre-infection treatments. Complete growth medium was added to both negative (no virus infection) and positive virus controls (virus infection without treatment). Cells were washed with fresh medium after 2-4 hours. To establish viral infection, frozen vials of HSV-1 or HSV-2 were thawed at 37° C. and kept on ice until desired virus dilutions were prepared in cold 1× MEM-5% FBS. I ml of indicated virus dilution was added per well after aspirating media, and the plates incubated at 37° C. for 1.5-2 hours. The virus-containing medium was aspirated and replaced with 2 ml of medium containing the respective test formulations. Post-infection treatments lasted from I to several days, with media replaced every 48 hours. Treated monolayers were examined microscopically, and following 0.5% crystal violet staining, and compared to control cultures to determine reduction of CPE using previously described methods (McLaren et al., 1983). For IC₅₀ studies, monolayers of Vero cells were infected at 10² PFU, but since BCD has limited aqueous solubility limiting our testing of higher concentrations, pooled human sera was added to the medium to a final concentration of 2%. The presence of pooled human sera limits the spread of cell-free virus, thereby limiting transmission of cell-associated virus to adjacent cells as the only means of plaque formation. Studies were done in triplicate and mean values used to calculate the IC₅₀ for BCD treatment of HSV-1.

Viral Growth Assays

Used media were harvested to measure the amount of cell-free virus present. 350 μl of culture medium collected at indicated times were placed on ice, frozen in liquid nitrogen and stored at −80° C. For cell-associated virus, infected cells were washed with Dulbecco's PBS, dissociated with trypsin, centrifuged, and resuspended in fresh medium. Cells were again centrifuged and resuspended in 700 μl growth medium before releasing the virus by three quick freeze-thaw cycles using liquid nitrogen and a 37° C. water bath. Virus lysates were cleared by centrifugation; supernatants containing virus were placed in liquid nitrogen for 2-3 minutes and stored at −80° C. for titration at a later date.

Infectious virus present in cell-free and cell-associated virus preparations obtained from the antiviral assays were titered by plaque assay in six-well plates utilizing a modified agarose overlay method (Dargan, 1998). Frozen samples were thawed, diluted, and 1 ml of virus dilutions applied per well after aspirating used medium. Plates were incubated at 37° C. for 1.5-2 hours. Virus dilutions were aspirated and 0.15 ml of fresh medium added per well. Immediately, 2 ml of a 1:1 dilution prepared from preheated 2% low melting temperature agarose in water (SeaPlaque® agarose BMA, Rockland, Me.) and 2× MEM-FBS held at 42° C. were added to each well. After gel formation, plates were incubated at 37° C. for 48 hours. 0.5 ml of a 1:100 dilution of 0.33% neutral red solution (Sigma) in 1×-MEM 5% FBS was added per well, and plates wrapped in aluminum foil. After 1 to 3 days of additional incubation at 37° C., plaques were visible as clear hollows over the reddish background. Counting was performed on wells containing 10 to 100 plaques.

Cell Viability Assay

Cell viability following BCD treatment was determined by performing flow cytometry using the Live/Dead® Viability/Cytotoxicity Assay Kit (L-3224, Molecular Probes, Eugene, Oreg.) and fluorometrically by LDH assay (Moran and Schnellmann, 1996). Vero cells were exposed to different formulations of BCD using 16 to 24 hour old confluent monolayers formed in T₇₅ flasks or 6-well plates. Used media were replaced with 15 ml/flask or 2.5 ml/well of treatment media, containing 5× MEM-FBS diluted to 1× with water (control), and □CD stock. After 48 hours, cells were collected, trypsinized, and resuspended in MEM containing 1 μM calcein and 4 μM ethidium homodimer-1 (EthD-1) and assayed for the percent live to dead cells on a flow cytometer (FACSalibur, BD Biosciences, San Jose, Calif.). Live cells are distinguished by the presence of intracellular esterase activity, as determined by the enzymatic conversion of calcein-AM (nonfluorescent) to calcein (green fluorescence). EthD-1 enters cells with damaged cell membranes and binds to nucleic acids producing red fluorescence to identify dead cells. EthD-1 is excluded from cells with intact plasma membranes (live cells). For LDH assays, cells were trypsinized, resuspended in fresh 1× medium, kept at 37° C.-5% CO₂ atmosphere, and total LDH present in cells and medium was determined after lysing the cells with 350 μM digitonin. The amount of LDH calculated for either condition was then compared with and expressed as a percentage of the total LDH present. Fresh 1× MFM-5% FBS was used as blank for all assays.

Effect of BCD on ACV-Resistant Virus

dlsptk is an ACV-resistant HSV-1 created by a deletion in the thymidine kinase gene, (Whitley and Roizman, 2001). BCD activity against dlsptk virus was determined using similar methods to those described above. BCD treated virus yield at 24 hours for HSV-1 KOS 1.1 and dlsptk strains were compared to ACV treated and untreated virus by the unpaired, student t-test using a p value <0.05 level of significance.

Effect of BCD on Replication Deficient Virus

d27-lacZ1 virus was treated with pre- and/or post-infection BCD and examined for β-gal activity. This virus has a deletion in the infected-cell protein (ICP27) gene, which encodes immediate-early (IE) genes that regulate viral gene expression. d27-lacZ1 is defective for lytic replication in Vero cells. V27 cells are Vero cells stably transfected with the ICP27 gene and allow multiple rounds of replication with d27-lacZ1 (Rice and Knipe, 1990). Following infection in Vero cells, wells were fixed chemically with 0.1% grade 1 glutaraldehyde (Sigma) solution in PBS for 5-10 minutes, washed three times with PBS, and overlaid with 1 ml/well of a 0.2 μm filtered cocktail of appropriate salinity containing X-gal, MgCl₂, ferri- and ferrocyanide. After several hours, the X-gal solution was aspirated, cells washed with PBS, and overlaid with 70% glycerol in water.

PCR Studies of HSV Entry

Effects of BCD on the yield of HSV DNA extracted from Vero cells infected with the recombinant β-gal HSV (d27-lacZ1) were investigated. Overnight confluent cultures were maintained in six-well plates or T₇₅ flasks and used media replaced with control or treatment medium containing BCD (final concentration 7.2 mg/ml). After 2 hours, the medium was aspirated and cells infected with d27-lacZ1 at an MOI of 0.01 or 0.1 for 2 hours. Virus dilutions were aspirated, mono-layers were exposed to pH 3 citrate buffer (40 mM sodium citrate, 10 mM KCl, and 135 mM NaCl in water) for 15-30 sec, and immediately washed with PBS 2-3 times. Control and test cells then received medium containing no test compound or 5.4 mg/ml of BCD. After 1 hour, cells were washed with PBS, harvested with trypsin, centrifuged, washed twice and finally resuspended in PBS. RNAase A (DNAase free) was added, and whole cell genomic DNA extracted following the manufacturer's recommendations (QIAmp® DNA Mini Kit, Qiagen, Valencia, Calif.). All DNA preparations were eluted in 400 μl, analyzed by agarose gel electrophoresis, and the OD was determined at A₂₆₀ and A₂₈₀.

PCR was performed on equal amounts of template DNA isolated from virus infected control versus BCD treated infected Vero cells to compare quantities of viral DNA in a semi-quantitative fashion. Conditions were optimized for PCR amplification of both viral and cellular DNA. Monkey β-actin primers were designed using a GenBank sequence (accession # AB004047) for cellular genomic DNA (forward primer, 5′-TGC TGT CCC TGT ACG CCT CT-3′ (SEQ ID NO: 1), and reverse primer, 5′-AGT CCA GGG CGA CAT AGC AC-3′ (SEQ ID NO:2). For HSV-1 detection, primers to glycoprotein B (gpB) were used: forward primer 5′-ATT CTC CTC CGA CGC CAT ATC CAC CAC CTT-3′ (SEQ ID NO:3), and reverse primer, 5′-AGA AAG CCC CCA TTG GCC AGG TAG T-3′ (SEQ ID NO:4).

Hotstart® PCR (Qiagen) was performed for 30 cycles on a System 2400 thermocycler (Perkin Elmer, Boston, Mass.) in 50 μl reactions containing 20 pmole of each primer using the following protocol: denaturation at 94° C. for 45 seconds, annealing at 60° C. for 60 seconds, and extension at 72° C. for 90 seconds, Electrophoresis was performed with 5 μl of PCR product loaded on 1.5% agarose gel.

Results

CD Antiviral Activity

BCD at a final concentration of 7-8 mg/ml, but not ACD (8 mg/ml), afforded almost complete protection of Vero cells against HSV-1 and HSV-2 infected at a MOI of 0.001 and 0.1 as assayed by inhibition of viral CPE. ACD antiviral activity was not studied further. The number of HSV-1 plaques formed per well following 48 hours of BCD treatment is shown in Table 3. The number of plaques listed in Table 3 is the mean of triplicate wells±the standard deviation. The number of HSV-1 plaques without BCD treatment was used as the 100% control level. Using this data, the IC₅₀ was determined to be 5.1 mg/ml of BCD for HSV-1. An IC₅₀ for BCD protection from HSV-2 was not determined. BCD is insoluble in water at concentrations above 10 mg/ml. BCD inhibited infection by HSV-1 (data not shown) and HSV-2 at a MOI of 2.0, as judged by direct microscopy and crystal violet staining. Combined pre- and post-infection treatment provided greater protection against HSV-1 or HSV-2, than post-infection treatment alone even at MOI>50 (data not shown). Similar results were obtained with BCD treatment using the d27-lacZ1 recombinant virus.

TABLE 3

| | BCD (mg/ml) | | | | | | |
|---|---|---|---|---|---|---|---|
| | 0 | 3.5 | 4.0 | 4.5 | 5.0 | 5.5 | 6.0 |
| Plaques ± | 139.7 ± 1.53 | 119.0 ± 1.0 | 111.0 ± 6.6 | 84.0 ± 10.53 | 74.67 ± 4.51 | 54.67 ± 5.68 | <10 |
| Std. Dev. | | | | | | | |
| % of | 100 ± 0.88 | 85.2 ± 0.58 | 79.47 ± 3.8 | 60.14 ± 6.08 | 53.46 ± 2.60 | 39.14 ± 3.28 | — |
| control ± | | | | | | | |
| SEM | | | | | | | |

Virus Assays

Yields of cell-free and cell-associated virus were markedly decreased following treatment with ACV, BCD or both compounds together at 24 hours post-infection of Vero cells with HSV-1 (FIG. 1). BCD alone, or BCD plus ACV was more effective at reducing virus numbers than ACV alone.

Cell Viability Assays

BCD treatment of Vero cells for 48 hours yielded a 91.23% cell viability (normalized to control cells) using 5.5 mg/ml of BCD, and 84.89% viability using 7.2 mg/ml BCD as determined by flow cytometry and the live/dead assay. Control cell viability was 93.58% (all studies performed in duplicate). LDH assay following 48 hours of treatment with 8.0 mg/ml of BCD yielded a cell viability of 68.57%.

BCD is Effective Against an ACV-Resistant Virus

BCD and ACV activity against non-resistant (KOS1.1) and ACV-resistant (dlsptk) strains of HSV-1 was found. At the compound formulations used, 4.5 μg/ml ACV reduced KOS1.1 yield at 24 hours by 10²-fold while 6.4 mg/ml BCD reduced virus yield by approximately 10³-fold. ACV showed no activity against the resistant strain, while BCD reduced ACV-resistant virus yield by approximately 10³ PFU.

BCD/ACV Combined Antiviral Activity

When using suboptimal concentrations of BCD (final concentrations of 4.5 or 4.95 mg/ml) in combination with ACV (final concentrations of 250-300 μg/ml), both compounds used in combination exhibited greater reduction of CPE caused by HSV-1 KOS1.1 strain than either compound alone.

Antiviral Effects of BCD Against d27-lacZ1 Recombinant Virus

BCD treatment of Vero cells infected with d27-lacZ1 β-gal recombinant virus showed almost complete virus protection as indicated by lack of β-galactosidase expression in treated-infected cells versus staining of untreated-infected cells. Pre- and post-infection treatment with BCD was more effective than post-infection treatment alone. In a parallel experiment, 400 μg/ml ACV did not affect β-galactosidase expression by d27-lacZ1, indicating that it does not prevent HSV IE gene expression.

HSV Entry Following BCD Treatment

Semiquantitative PCR studies of HSV-1 DNA from BCD treated versus untreated infected Vero cells were conducted. Bands from treated and untreated infected cells appeared similar, indicating BCD did not significantly alter viral entry. Treated and control cells were each infected at a MOI of 0.01 or 0.1 and demonstrated comparable levels of viral gpB PCR product following amplification of stepwise increments of extracted DNA compared to cellular β-actin PCR product. To confirm the methodology used herein, additional PCR was performed on initial products. At low MOI, increased increments of product from previous PCR produced heavier bands, while at high MOI, bands appeared more or less uniform indicating linear and plateau phases of PCR amplification, respectively.

Discussion

Herpes simplex virus causes persistent and recurring human infections accounting for significant morbidity and mortality in immunocompetent and immunosuppressed patients (Whitley and Roizman, 2001). Acyclovir and other nucleoside analogs have been used successfully to reduce morbidity and transmission during active infection, but because of frequent ACV resistance, particularly in immunocompromised patients, newer antiviral therapies are needed (Shin et al., 2001; Field, 2001). Recent reports have documented antiviral activity of cyclodextrins, particularly substituted or charged beta-cyclodextrins (Leydet et al., 1998; Liao et al., 2001). The activity of ACD and BCD against HSV-1 and HSV-2 was studied. ACD exhibited no significant antiviral activity against HSV-1 or HSV-2 in infected Vero cells at final concentrations up to 8 mg/ml, while BCD showed significant anti-HSV-1 and HSV-2 activity even at very high MOI.

Cyclodextrins contain 6 (α-CD), 7 (β-CD) or 8 (γ-CD) dextrose subunits with aqueous solubility (Irie and Uekama, 1997; Loftsson and Masson, 2001; Loftsson, 1999; Rajewski and Stella, 1996). Because of a lipophilic central cavity, CD are capable of complexing with a number of agents with more limited water solubility to form drug-CD complexes. Substitution of the external hydroxyl groups may increase their aqueous solubility (Rajewski and Stella, 1996). CD have variable, but generally low toxicity in laboratory animals, cell culture and humans, in part because they have a low capability of penetrating cell membranes and have low oral absorption secondary to degradation by intestinal bacteria (Bellinger et al., 1995; Irie and Uekama, 1997; Loftsson and Masson, 2001). BCD has the lowest aqueous solubility of natural CD, and forms crystal lattice precipitates in aqueous solutions with several complexes (Bellinger et al., 1995; Loftsson and Masson, 2001). Crystal formation does not appear to significantly alter BCD toxicity or efficacy. CD have been investigated or are used as drug carriers for a number of medicinals including antimycotic agents, anti-inflammatory agents, steroids, prostaglandins, retinoids, hormones, or as nucleic acid carriers for gene therapy (Bellinger et al., 1995; Irie and Uekama, 1997; Loftsson and Masson, 2001; Pedersen et al., 1999; Rajewski and Stella, 1996). The antiviral mechanism of action of BCD is unknown. CD are known to cause cholesterol efflux from membranes, which may in part, be responsible for their antiviral activity (Kilsdonk et al., 1995; Ohtani et al., 1989). Potassium efflux from erythrocytes has also been observed from studies of CD treated erythrocytes (Ohtani et al., 1989). CD may also alter the sphingolipid domains of murine lymphocyte and endothclial membranes with the efflux of glycosylphosphatidylinositol (GPI)-anchored proteins (Ilangumaran and Hoessli, 1998; Kilsdonk et al., 1995). This may alter either cell membrane receptors or signal transduction pathways.

Recent reports have documented in vitro activity of BCD and CD derivatives against HIV (Leydet et al., 1998; Liao et al., 2001). Liao et al. showed 2-OH-propyl-β-cyclodextrin in vitro activity against HIV-1, and determined that this activity was likely due to cholesterol depletion of lipid rafts, important to virus budding and synctium formation (Liao et al., 2001). Leydet et al. investigated the antiviral activity of several charged CD derivatives and demonstrated anti-HIV and anti-CMV activity, but found no anti-HSV activity with the CD derivatives and parameters utilized (Leydet et al., 1998). They used a number of CD derivatives at different concentrations and in different cell lines than were utilized in the current study. As disclosed herein, the unsubstituted form of BCD provides almost complete protection to Vero cells against CPE caused by HSV-1 or HSV-2 infection even at very high MOI. BCD reduced both cell-free and cell-associated virus more effectively than ACV at the concentrations used indicating a reduction of viral replication and/or entry. BCD did exhibit cytoxicity to Vero cells as determined by live/dead and LDH release assays, but this effect was seen largely after 48 hours of BCD treatment and at higher concentrations than necessary to demonstrate antiviral activity. Therefore, the reduction in plaque numbers of BCD treatment is not an artifact of cytotoxicity.

Significantly, BCD had marked anti-HSV-1 activity against an ACV-resistant strain. This suggests that BCD works at a different step of virus replication, which could have major clinical significance. Furthermore, sub-optimal concentrations of BCD in combination with ACV showed an additive protective effect when Vero cells were infected with the KOS1.1 strain. Unlike ACV, which interferes with late stages of viral replication, BCD appears to interfere with a stage(s) prior to IE gene expression of HSV-1, as demonstrated by treatment of d27-lacZ1 recombinant virus-infected Vero cells. PCR studies of BCD treated HSV-1 infected Vero cells indicate BCD probably does not diminish cell entry of the virus.

In conclusion, (i) BCD has potent in vitro antiviral activity against HSV-1 and HSV-2 in Vero cells, (ii) the mechanism of action of BCD is different than that of ACV, (iii) BCD is effective against an ACV-resistant strain of HSV-1, and that (iv) BCD appears to exert its mechanism of action at an early stage of viral replication.

### REFERENCES

Aoki, Herpes, 8, 41-45 (2001).
Bellinger et al., Food Chem Toxicol, 33, 367-376 (1995).
Blight et al., 2000; Science: 290: 1972).
Bourne et al., Antiviral Res, 40, 139-144 (1999).
Cates, Sexually Transmitted Diseases, 26, s2-s7 (1999).
Coen et al., Proc. Natl. Acad. Sci. U.S.A., 86, 4736-4740 (1989).
Constance, “Preparation of a Specific Retrovirus Producer Cell Line: X-gal Staining of Cultured Cells, Unit 9.10.9,” In Ausubel et al. (eds.), Current Protocols in Molecular Biology, Vol. 2, supplement 36. John Wiley and Sons, Inc., New York, N.Y. (1995).
Crumpacker, “Antiviral Therapy,” p. 393-⁴33, In Knipe and Howley (eds.), Fields Virology, 4th ed. Vol. 1, Lippincott Williams & Wilkins Publishers, Philadelphia, Pa. (2001).
Dargan, Investigation of the anti-HSV activity of candidate antiviral agents: in Brown S M, MacLean A R (ed): Methods in Molecular Medicine. Humana Press, Inc. Totowa, N.J., 1998, pp 387-405.
Elion et al., Proc Natl Acad Sci, 74, 5716-5720 (1977).
Esposito and Fenner, “Poxviruses,” p. 2885-2921, In Knipe and Howley (eds.), Fields Virology, 4th Ed. Vol. 2, Lippincott Williams & Wilkins Publishers, Philadelphia, Pa. (2001).
Federoff, “Replication-defective Herpesvirus Amplicon Vectors and Their Use for Gene Transfer,” p 91.1-91.10, In Spector et al. (eds.), Cells: A Laboratory Manual, 2nd Vol., Cold Spring Harbor Laboratory Press, New York, N.Y. (1998).
Field, J Clin Virol, 21, 261-269 (2001).
Howley and Lowy, “Papillomaviruses and Their Replication,” p. 2197-2230, In Knipe and Howley (eds.), Fields Virology, 4th ed. Vol. 2, Lippincott Williams & Wilkins Publishers, Philadelphia, Pa. (2001).
Hughes and Munyon, J Virol, 16, 275-283 (1975).
Ilangumaran and Hoessli, Biochem J, 335, 433-440 (1998).
Irie and Uekama, J Pharmacet Sci 86 147-162 (1997).
Jay and Moscicki, “Human Papilloma Virus Infection in Women”, In: Women & Health, Goldman and Hatch, (eds.), San Diego, Calif.: Academic Press. (2000).
Kaiser Family Foundation, “National Survey of Public Knowledge of HPV, the Human Papillomavirus”, available on the world wide web at kff.org/content/2000/20000217a/HPVChartpack2 PDF (2000).
Khan et al., “In vitro activity of beta-cyclodextrin against HSV-1 and HSV-2,” manuscript submitted to Antimicrob. Chemother. (March 2003).
Khanna et al., The Journal of Clinical Investigation, 109, 205-211 (2002).
Kilsdonk et al., J Biol Chem, 270 17250-17256 (1995).
Leydet et al., J Med Chem, 41, 4927-4932 (1998).
Liao et al., AIDS Res. Hum. Retroviruses, 17, 1009-1019 (2001).
Loftsson and Masson, Int J Pharmaceut, 225, 15-30 (2001).
Loftsson, Pharm Technol Eur, 11, 20-32 (1999).
Lowy and Howley “Papillomaviruses,” p. 2231-2264, In Knipe and Howley (eds.), Fields Virology, 4th ed. Vol. 2, Lippincott Williams & Wilkins Publishers, Philadelphia, Pa. (2001).
Major et al. “Hepatitis C Viruses,” p. 1127-1162, In Knipe and Howley (eds.), Fields Virology, 4th ed. Vol. 1, Lippincott Williams & Wilkins Publishers, Philadelphia, Pa. (2001).
MeLaren et al., Antiviral Res, 3, 223-234 (1983).
Mertz, Herpes simplex virus infections; in Galasso G J, Whitley R J, Merigan T C (ed): Antiviral agents and human viral diseases. Lippincott-Raven. Philadelphia, 4th edition, 1997, pp 305-341.
Miller et al. Fertil. Steril., 57,1126-28 (1992).
Mocarski and Courcelle, “Cytomegaloviruses and Their Replication,” p. 2629-2674, In Knipe and Howley (eds.), Fields Virology, 4th ed. Vol. 2, Lippincott Williams & Wilkins Publishers, Philadelphia, Pa. (2001).
Moran and Schnellmann, J Pharmacol Toxicol Methods, 36, 41-44 (1996).
Naesens and De Clercq, Herpes, 8 12-16 (2001).
Nicolazzi C, Venard V, Le Faou A, Finance C. In vitro antiviral efficacy of the ganciclovir complexed with β-cyclodextrin on human cytomegalovirus strains. Antiviral Res 2002;54:121-127.
Ohtani et al., Eur J Biochem, 186, 17-22 (1989).
Pass “Cytomegalovirus,” p. 2675-2706, In Knipe and Howley (eds.), Fields Virology, 4th ed. Vol. 2, Lippincott Williams & Wilkins Publishers, Philadelphia, Pa. (2001).
Pedersen et al., J Drug Develop & Ind Pharm, 25, 463-470 (1999).
Rajewski and Stella, J Pharmaceut Sci, 85, 1142-1169 (1996).
Ramakrishnan et al. J. Virol., 68,1864-73 (1994).
Rice and Knipe, J Virol, 64, 1704-1715 (1990).
Schacker, Herpes, 8, 46-49 (2001).
Schnipper and Crumpacker, Proc Natl Acad Sci, 77, 2270-2273 (1980).
Shin et al., J Clin Microbiol, 39, 913-917 (2001).
Taylor et al., Front Biosci, 7, d752-d764 (2002).
Tebas et al., J Infect Dis, 177, 217-220 (1997).
Whitley and Roizman, Lancet, 357, 1513-1518 (2001).
Zeitlin L, Whaley K J, Hegarty T A, Moench T R, Cone R A. Tests of vaginal microbicides in the mouse genital herpes model. Contraception 1997;56:329-335.

All publications, patents, and patent documents are incorporated by reference herein, as though individually incorporated by reference. The invention has been described with reference to various specific and preferred embodiments and techniques. However, it should be understood that many variations and modifications may be made while remaining within the spirit and scope of the invention.

1-13. (canceled)

14. A pharmaceutical composition comprising a therapeutically effective anti-viral amount of a cyclodextrin or a pharmaceutically acceptable salt thereof:
wherein the cyclodextrin is unsubstituted or substituted at one or more —OH moieties with an —OR moiety, R comprising a linear or branched alkylene moiety having from 1 to 6 carbon atoms, or a hydroxyalkylene moiety; and
wherein the cyclodextrin is not in the form of an inclusion complex with another compound;
with the proviso that when the cyclodextrin is substituted, the cyclodextrin does not comprise a polyanionic substituted cyclodextrin.

15. The pharmaceutical composition according to claim 14, wherein the cyclodextrin comprises α-cyclodextrin.

16. The pharmaceutical composition according to claim 14, wherein the cyclodextrin comprises β-cyclodextrin.

17. The pharmaceutical composition according to claim 14, wherein the cyclodextrin comprises γ-cyclodextrin.

18. The pharmaceutical composition according to claim 14, wherein the cyclodextrin is selected from the group consisting of methyl cyclodextrin, ethyl cyclodextrin, hydroxypropyl cyclodextrin, hydroxyethyl cyclodextrin, and sulfobutyl ether cyclodextrin.

19. The pharmaceutical composition according to claim 14, wherein the cyclodextrin is selected from the group consisting of hydroxypropyl-α-cyclodextrin, hydroxypropyl-β-cyclodextrin, and hydroxypropyl-γ-cyclodextrin.

20. The pharmaceutical composition according to claim 14, wherein the cyclodextrin comprises 2-hydroxypropyl-β-cyclodextrin.

21. The pharmaceutical composition according to claim 14, wherein the cyclodextrin or pharmaceutically acceptable salt thereof comprises about 2 wt-% to about 60 wt-% of the composition.

22. The pharmaceutical composition according to claim 14, wherein the cyclodextrin or pharmaceutically acceptable salt thereof comprises about 0. 1 wt-% to about 25 wt-% of the composition.

23. The pharmaceutical composition according to claim 22, wherein the cyclodextrin or pharmaceutically acceptable salt thereof comprises about 0.5 wt-% to about 10 wt-% of the composition.

24. The pharmaceutical composition according to claim 14, wherein the composition comprises a form for topical administration selected from the group consisting of lotion, cream, gel, and ointment.

25. The pharmaceutical composition according to claim 24, wherein the composition is in the form of a gel.

26. The pharmaceutical composition according to claim 14, wherein the composition is in the form of an inhalant.

27. The pharmaceutical composition according to claim 14, wherein the composition is in the form of a solid or semi-solid.

28. The pharmaceutical composition according to claim 14, said composition specifically excluding any further anti-viral active agents.

29. The pharmaceutical composition according to claim 14, said composition specifically excluding any further active agents.

30. A pharmaceutical composition comprising a cyclodextrin or a pharmaceutically acceptable salt thereof in a therapeutically effective anti-viral amount of about 2 wt-% to about 60 wt-%, wherein the cyclodextrin is unsubstituted or substituted at one or more —OH moieties with an —OR moiety, R comprising a linear or branched alkylene moiety having from 1 to 6 carbon atoms, or a hydroxyalkylene moiety, and with the proviso that when the cyclodextrin is substituted, the cyclodextrin does not comprise a polyanionic substituted cyclodextrin.

31. The pharmaceutical composition according to claim 30, wherein the cyclodextrin comprises α-cyclodextrin.

32. The pharmaceutical composition according to claim 30, wherein the cyclodextrin comprises β-cyclodextrin.

33. The pharmaceutical composition according to claim 30, wherein the cyclodextrin comprises γ-cyclodextrin.

34. The pharmaceutical composition according to claim 30, wherein the cyclodextrin is selected from the group consisting of methyl cyclodextrin, ethyl cyclodextrin, hydroxypropyl cyclodextrin, hydroxyethyl cyclodextrin, and sulfobutyl ether cyclodextrin.

35. The pharmaceutical composition according to claim 30, wherein the cyclodextrin is selected from the group consisting of hydroxypropyl-α-cyclodextrin, hydroxypropyl-β-cyclodextrin, and hydroxypropyl-γ-cyclodextrin.

36. The pharmaceutical composition according to claim 30, wherein the cyclodextrin comprises 2-hydroxypropyl-β-cyclodextrin.

37. The pharmaceutical composition according to claim 30, wherein the composition comprises a form for topical administration selected from the group consisting of lotion, cream, gel, and ointment.

38. The pharmaceutical composition according to claim 30, wherein the composition in the form of an inhalant.

39. The pharmaceutical composition according to claim 30, wherein the composition is in the form of a solid or semi-solid.

40. The pharmaceutical composition according to claim 30, further comprising one or more additional anti-viral agents.

41. The pharmaceutical composition according to claim 30, wherein the one or more additional anti-viral agents are selected from the group consisting of acyclovir, foscamet, famciclovir, penciclovir, and valacyclovir.

42. A pharmaceutical composition comprising about 2 wt-% to about 60 wt-% of a cyclodextrin selected from the group consisting of methyl cyclodextrin, ethyl cyclodextrin, hydroxypropyl cyclodextrin, and hydroxyethyl cyclodextrin.

43. The pharmaceutical composition according to claim 42, wherein the composition comprises a form for topical administration selected from the group consisting of lotion, cream, gel, and ointment.

44. The pharmaceutical composition according to claim 42, wherein the composition in the form of an inhalant.

45. The pharmaceutical composition according to claim 42, wherein the composition is in the form of solid or semi-solid.

46. The pharmaceutical composition according to claim 42, further comprising one or more additional anti-viral agents.

47. The pharmaceutical composition according to claim 42, wherein the one or more additional anti-viral agents are selected from the group consisting of acyclovir, foscamet, famciclovir, penciclovir, and valacyclovir.

48. A pharmaceutical composition for topical administration, said composition comprising:
(a) a cyclodextrin or a pharmaceutically acceptable salt thereof in a therapeutically effective anti-viral amount of about 2 wt-% to about 60 wt-%, wherein the cyclodextrin is unsubstituted or substituted at one or more —OH moieties with an —OR moiety, R comprising a linear or branched alkylene moiety having from 1 to 6 carbon atoms, or a hydroxyalkylene moiety; and
(b) a dermatologically acceptable carrier.

49. The pharmaceutical composition according to claim 48, wherein the dermatologically acceptable carrier comprises a solid carrier.

50. The pharmaceutical composition according to claim 48, wherein the dermatologically acceptable carrier comprises a liquid carrier.

51. The pharmaceutical composition according to claim 48, wherein the cyclodextrin comprises a-cyclodextrin.

52. The pharmaceutical composition according to claim 48, wherein the cyclodextrin comprises β-cyclodextrin.

53. The pharmaceutical composition according to claim 48, wherein the cyclodextrin comprises γ-cyclodextrin.

54. The pharmaceutical composition according to claim 48, wherein the cyclodextrin is selected from the group consisting of methyl cyclodextrin, ethyl cyclodextrin, hydroxypropyl cyclodextrin, hydroxyethyl cyclodextrin, and sulfobutyl ether cyclodextrin.

55. The pharmaceutical composition according to claim 48, wherein the cyclodextrin is selected from the group consisting of hydroxypropyl-α-cyclodextrin, hydroxypropyl-β-cyclodextrin, and hydroxypropyl-γ-cyclodextrin.

56. The pharmaceutical composition according to claim 48, wherein the cyclodextrin comprises 2-hydroxypropyl-β-cyclodextrin.

57. The pharmaceutical composition according to claim 48, wherein the cyclodextrin is not in the form of an inclusion complex with another compound.

58. The pharmaceutical composition according to claim 48, wherein the composition in the form of a gel.

59. The pharmaceutical composition according to claim 48, wherein the composition in the form of a solid or semi-solid.

60. The pharmaceutical composition according to claim 48, wherein the composition in the form of an inhalant.

61. The pharmaceutical composition according to claim 48, further comprising one or more additional anti-viral agents.

62. The pharmaceutical composition according to claim 48, wherein the one or more additional anti-viral agents are selected from the group consisting of acyclovir, foscamet, famciclovir, penciclovir, and valacyclovir.


SEQUENCE LISTING (4 sequences)

SEQ ID NO: 1 - DNA, 20 bp, Cercopithecus aethiops
tgctgtccctgtacgcctct

SEQ ID NO: 2 - DNA, 20 bp, Cercopithecus aethiops
agtccagggcgacatagcac

SEQ ID NO: 3 - DNA, 30 bp, Herpes Simplex Virus
attctcctccgacgccatatccaccacctt

SEQ ID NO: 4 - DNA, 25 bp, Herpes Simplex Virus
agaaagcccccattggccaggtagt